(19) United States
(12) Patent Application Publication
(10) Pub. No.: US 2012/0045748 A1
(43) Pub. Date: Feb. 23, 2012

(54) Particulate labels

(76) Inventors: Richard C. Willson, Houston, TX (US); Ulrich Strych, Pearland, TX (US); Binh V. Vu, Houston, TX (US)

(21) Appl. No.: 13/135,364

(22) Filed: Jun. 30, 2011

Related U.S. Application Data
(60) Provisional application No. 61/398,717, filed on Jun. 30, 2010.

(51) Int. Cl. C12Q 1/70; C12Q 1/68

(52) U.S. Cl. 435/5; 435/6.11; 435/6.12

(57) ABSTRACT

A methodology for bioassays and diagnostics in which a particulate label (ranging in size from nm-scale molecular assemblages to organisms on the scale of tens or hundreds of microns), such as, but not limited to, nanoparticles, bacteria, bacteriophage, Daphnia, and magnetic particles, serve carriers for analytes bound by molecular recognition elements such as antibodies, aptamers, etc. The described methodology is generally applicable to most pathogen assays and molecular diagnostics and also leads to enhanced sensitivity and convenience of use.

### CROSS-REFERENCE TO RELATED APPLICATIONS

This application claims the benefit of U.S. provisional Ser. No. 61/398,717, filed Jun. 30, 2010 by the present inventors.

### STATEMENT REGARDING FEDERALLY SPONSORED RESEARCH OR DEVELOPMENT

Not applicable.

### REFERENCE TO A SEQUENTIAL LISTING

Not Applicable.

### FIELD OF THE INVENTION

The present invention relates generally to chemical analysis, and more particularly, to assays for biological analytes using particulates as an element of the assay method.

### BACKGROUND OF THE INVENTION

The detection of chemical analytes, including toxins and industrial chemicals as well as biological molecules, cells, viruses, and pathogens, is of great importance in modern society. Environmental health and safety, chemical and biological defense, sample identification, biomedical investigations, and medical diagnostics all depend upon reliable detection and quantitation of chemical and biological species and organisms.

Biological research and medical practice are particularly dependent upon methods of detecting and quantitating molecules, viruses and cells. Of particular importance are the detection of pathogens such as bacteria, parasites, and viruses, and the detection of proteins and nucleic acids, among other examples identified in Table 1.

Detection and quantitation of these types of analytes is increasingly important in the investigation of biological processes, including in areas known as proteomics, genomics, epigenetics, and interactomics.

Practical applications include diagnosing infections with pathogenic cells and viruses, protecting against bioterrorism, and diagnosing infectious diseases. Specific biomarkers, including microRNAs, proteins and modified proteins are useful in diagnosing cancer, in choosing which therapeutic drugs to use, in detecting relapse, and in identifying the appearance of drug resistance among other examples identified in Table 1.

A very large range of organisms, viruses, and chemical species, collectively referred to as analytes, are of interest in modern science, technology and medicine. Illustrative examples of these are listed in Table 1, which does not constitute a complete listing. There is a felt need for detection and analysis methods combining desirable characteristics such as high sensitivity, convenience and reliability, low cost, speed, and/or the ability to be performed in parallel on multiple analytes.

The analytical method to be employed depends, in part, on the origins of the species to be detected and the example within which they are to be detected. Some examples are listed in Table 1, and include medical specimens, environmental samples, and food.

The overall analytical process nearly always includes some sample-preparation steps using various sample preparation agents, some of each of which are illustrated in Table 1. These may include, for example, concentration of a dilute species from a liquid or gaseous environment using a filter, isolation of a subset of cells from a complex blood sample, breakage of cells to liberate analytes of interest, or removal of lipids and particulates which could interfere with later analysis.

In addition to concentrating, enriching, and/or partially-purifying the analytes of interest, in some cases, it is possible to achieve amplification of the analyte to be detected, for example, by the use of polymerase chain reaction to amplify nucleic acids or nucleation chain reaction to amplify prion proteins, or growth of an organism. Another way to amplify the detectability of a label is to grow an assembly of biomolecules, such as an actin filament or immune complex. Another is to use a nucleating agent (eg, of bubbles, crystals or polymerization) as an element of the label. Where available, these methods can greatly facilitate subsequent analysis.

Many analytical methods, including those of interest in the present invention, involve molecular recognition, and also transduction of the molecular recognition event into a usable signal. Molecular regulation refers to the high affinity and specific tendency of particular chemical species to associate with one another, or with organisms or viruses displaying target chemical species. Well-known examples of molecular recognition include the hybridization of complimentary DNA sequences into the famous double helix structure with very high affinity, and the recognition of foreign organisms or molecules in the blood stream by the antibodies produced by mammals, or selected analytes by deliberately selected monoclonal antibodies.

As partially listed in Table 1, there are many other examples of molecular recognition elements, including the recognition of carbohydrate molecules by lectins, nucleic acid recognition by proteins and nucleic acid analogs, the binding of analytes by antibody fragments, derivatives, and analogs, and a host of other examples.

A complete method of detection and analysis requires, in addition to molecular recognition, some means of reading out of molecular recognition event into a usable signal. This reading-out or transduction is the main focus of the present invention. Because of the importance of detection, analysis, and quantitation of chemical and biological species, the prior art contains many examples of technologies for carrying out these analyses. The prior art technologies mostly employ conventional molecular recognition elements, especially antibodies and nucleic acids, and have varied in the means of transducing molecular recognition into a useful signal.

In particular, successive generations of means of labeling antibodies and nucleic acids so that their binding to a target analyte may be more easily detected have shaped large portions of the field for decades. Successive generations of these types of assays have involved immobilizing the target analyte onto a solid planar surface, typically a membrane or the flat bottom of a microtiter plate well, either by non-specific absorption or by antibody capture in most cases. Then a labeled molecular recognition element such as a nucleic acid probe or antibody is added and allowed to bind to the immobilized analyte. After washing, the label is detected and the presence of the label is used to infer the presence of the analyte on the surface, and therefore in the original sample.

Labels have included radioactive isotopes, enzymes with reactive substrates capable of generating absorbance at specific wavelengths, light or fluorescence, or fluorescent molecules directly coupled to the molecular recognition agent. Absorbance has typically been measured in liquids in wells 1 mm to 1 cm across, and across path lengths of 1 mm to 1 cm. Recent scientific findings that even sub-100 nm orifices in metal films can support detectable transmission of light (“extraordinary optical transmission”) has led to great activity in observing shifts in the wavelength distribution, the color, of light passing through such orifices in response to the presence of analyte (see, for example, Yanik et al., 2010). What has not been exploited is that the phenomenon of extraordinary optical transmission supports the present invention that blockage or reduction of light passing through small orifices may allow very sensitive detection of analytes. This blockage can be achieved by decorating the surface containing the orifices with a molecular recognition agent, and capturing analyte which may strongly, directly block light transmission, or allow staining (e.g., with particles or enzyme products such as silver or BCIP/NBT products) with a strongly light-blocking material. Of particular interest in this application is the use of orifices of the size of single cells, viruses or molecules. It should be noted that this assay format also can be inverted in geometry to use a larger light source, and small detectors such as optical fibers.

A related aspect of this innovation is the blockage of electrical current flowing through submicron orifices or tubes by particles which adhere to a molecular recognition element on the wall of the orifice or tube when analyte is present, or whose current-blocking properties are changed by the capture of analytes and optionally stains onto their surfaces via immobilized molecular recognition elements. While the Coulter principle of detecting and counting particles as they pass through a large orifice has long been known, it has not been applied in these ways to detection of smaller analytes by observation of the behavior of larger particles, and also has never been applied on such a small scale.

One Coulter-principle-based commercial device useful in practicing the present invention is made by IZON, inc.; this machine has the advantage of adjustable orifice size, and is most readily suitable for particles in the range of 150 nm to a few microns. It measures particle number density, size (by degree of current reduction), and electrophoretic mobility, which relates both to size and charge and is reported by blockade duration. Of particular utility with this machine are changes in the charge and conductivity of particles, and the capture of particles in or near the orifice by molecular recognition agents located on the orifice material. It is preferable that particles, cells, viruses or other labels used in the IZON machine be relatively uniform to facilitate detection of changes; it is more preferred that the standard deviation of size be less than 10%. This issue favors the use of particles which have been pre-fractionated for uniformity (e.g. by settling, chromatography, or field-flow fractionation), or produced in a way that yields uniform particles (e.g., lithography, or the biological formation of phage, viruses, or spores).

Particles also can be used to support assays based on analyte-dependent changes in vibrational motions responsive to mass. An aspect of this invention is the change of the vibrational behavior of a vibrating element having a channel inside it, by particles which adhere to a molecular recognition element on the wall of the channel when analyte is present, or whose buoyant masses are changed by the capture of analytes and optionally stains onto their surfaces via immobilized molecular recognition elements. While the quartz crystal microbalance detection of analytes adsorbing to the surface of a vibrating crystal has long been known, and the Archimedes vibrating cantilever microbalance achieves great sensitivity in weighing nanoparticles, they have not been applied in these ways to detection of smaller analytes by observation of the behavior of larger particles, and also QCM has never been applied on such a small scale. At least equally novel is the present detection of analyte binding by the actual weighing of individual particles bearing analyte and optionally stains such as nanoparticles, phage, Polymerase Chain Reaction (PCR) products or silver deposits.

As noted above, one vibrating mass sensor-based commercial device useful in practicing the present invention is the Archimedes made by NN, inc., which uses a microfabricated vibrating cantilever containing a flow channel as a mass sensor. Mass can be detected when it become associated with the channel walls, or as increases in the total mass in the channel when particles of density different from the (adjustable) density of the running fluid. Particles of diameter below about 4 microns currently are favored to avoid settling and channel clogging, though channels of different sizes are available to accommodate particles of varying size. There is no specific limit for particle size, as the detection limit is set by mass and higher-density particles can be detected at smaller sizes. The current lower limit is near 5 fg, though improvements are expected, and gold nanoparticles of 50 nm diameter can be used. Lower-density particles often are preferred because they offer a larger surface area for detectable binding and staining events to occur, with minimal starting mass (against which changes are to be detected). Similarly, label materials to be captured or deposited by staining are preferably of high density, such as gold or other metal nanoparticles, silver deposits, and uranium-based stains. Of particular utility with this machine are changes in the mass of particles, and the capture of particles in or near the orifice by molecular recognition agents located on the channel walls. It is preferable that particles, cells, viruses or other labels used in the Archimedes machine be relatively uniform to facilitate detection of changes; it is more preferred that the standard deviation of size be less than 10%. This issue favors the use of particles which have been pre-fractionated for uniformity (e.g. by settling, chromatography, or field-flow fractionation), or produced in a way that yields uniform particles (e.g., lithography, or the biological formation of phage, viruses, or spores).

These types of solid-phase binding assays have been enormously useful and influential and are widely practiced to this day. They suffer in some cases from a lack of sensitivity, from the relatively laborious steps involved in successive binding and washing (complicated by the difficulties of mass-transfer to the solid phase).

Enormous sensitivity is often achieved in assays which detect nucleic acids. These assays usually are based on the specific recognition of DNA sequences via hybridization, often coupled with amplification by the action of polymerases, ligases, helicases, and other enzymes. Affinity can be increased by the use of chemical modifications such as peptide or locked nucleic acids, or groove-binding agents. Nucleic acid elements of labels are best chosen depending on the intended method of detection. For example, detection by molecular beacon hybridization or enzyme-labeled probe capture can use relatively short RNA or DNA sequences, down to potentially as low as 10 nucleotides but preferably longer than 20 nucleotides, but the lower sensitivity of these methods will generally require that more copies of the target be provided. Polymerase chain reaction, by contrast, requires a DNA template of at least 30 nucleotides, preferably 50 nucleotides, and in many cases ideally over 200 nucleotides. PCR, however, offers enormous sensitivity down to a few or even one copy of the template target sequence. DNA and RNA sequences often are preferably librated from association with other moieties before detection, as by the breakage of liposomes, reduction of redox-sensitive couplings, protease treatment, or digestion with a specific nuclease such as a restriction enzyme.

PCR and many of the related assays are limited to detection of nucleic acids. One particularly sensitive class of assays which is not nearly as popular as might be expected combines analyte binding by antibodies with PCR detection of a DNA sequence physically associated with the antibody.

The combination of antibody molecular recognition with DNA amplification was first described by Sano et al., who coined the term “Immuno-PCR” in 1992⁵. In immuno-PCR, antibody-DNA conjugate was used in place of antibody-enzyme conjugate. Antibody is conjugated to biotinylated DNA via a recombinant chimera consisting of protein A and streptavidin. Immuno-PCR has been shown be more sensitive than enzyme immunoassay procedures⁶⁻⁹. However, immuno-PCR is not widely adopted because of the demanding protocols¹⁰ and complexity in antibody-DNA conjugation¹¹. The use of protein A-streptavidin chimera is limited to direct assay format, which does not have capture antibodies¹². Thus, multiplexing, the ability to simultaneously detect multiple antigens, is not possible. Another problem with the original immuno-PCR method is the issue with high background due to non-specific binding of antibody-DNA conjugate to assay microplate¹⁰. One aspect of the present invention addresses this non-specific binding by the use of polycationic compounds, especially spermine and spermidine, to reduce the very high charge density of antibody-DNA conjugates.

As an alternative to protein A-streptavidin chimera, a chemical coupling method was developed which used a hetero-bifunctional crosslinking agent¹³. However, antibody stability was significantly compromised because of the harsh condition in additional chromatography purification steps¹⁰. To address these problems, in 2002, Nam et al. introduced nanoparticle-based bio-bar codes which used gold nanoparticles as the linkers for both DNA and antibody¹⁴. In this approach, DNA is thiolated and immobilized to gold nanoparticles through thiol-gold chemistry. Antibody, on the other hand, is immobilized to the same gold nanoparticles by passive adsorption. This method also utilizes magnetic microparticles with immobilized antibody to capture antigen, but did not provide a force-mediated stringency to reduce non-specific adsorption, which is an element of the present invention.

In 2007, Wacker et al. reported another method which used antibody immobilized bacterial magnetosome instead of magnetic particles to capture antigen. The detection was done with chemically coupled antibody-DNA conjugate¹⁵. Also in 2007, Burbulis et al. reported an innovative method for DNA-antibody conjugation using intein-mediated ligation to ligate cysteine DNA to an intein protein G fusion¹¹. There have been other variations of the above methods, however, the DNA-antibody conjugates were prepared by the basis linkers: streptavidin-biotin, hetero-bifunctional chemical linkers, or gold nanoparticles¹⁶⁻¹⁷. As an element of this invention, we have developed a novel method of preparing conjugates of a molecular recognition element with DNA in which the label DNA sequence is fused to an aptamer which recognizes the molecular recognition agent in such a way as not to interfere with its binding to the analyte of interest. These linked molecules are broadly useful, can have greater antibody type specificity than Protein A (permitting sandwich assays), are easy to prepare, and can bear multiple copies of the tag sequence though repetition or branching of the label DNA.

In an improvement of the basic concept of immuno-PCR, we have developed a novel method which used magnetic nanoparticles as DNA and antibody carriers. The magnetic property of DNA-antibody carrier has significant benefits in reagent preparation and background reduction in assay. Because of their magnetic property, washing of magnetic nanoparticles containing both DNA and antibody is easily performed, and the use of magnetic pull-off to enhance stringency produces lower background and higher useful sensitivity than other methods. Since protein A and other antibody binding proteins are not used, this method also can be used in sandwich format, thus making multiplexing possible.

One final way to associate detectable DNA (or RNA) with a molecular recognition element is to include the DNA in an organism bearing a molecular recognition element on its surface. This type of surface display is widely used to screen libraries of molecular recognition elements for binding to a preselected target, by displaying them on the surface of viruses, phage, bacteria, yeast, or spores, or associated with ribosomes, usually after mutagenesis. As is widely known, the libraries are then allowed to bind to surfaces or particles, bound and unbound members of the library are separated from each other, and binders iteratively enriched and selected to isolate molecular recognition elements capable of binding the preselected target. Negative selection for non-binders is also known, and an element of the present invention is the use of cells or viruses which have been negatively selected against non-specific binding as elements of labels for assays.

As an element of this invention, we provide four major improvements of this approach, some of which are applicable to other methods taught here. The first is the use of phage or other cells or viruses genetically selected for low non-specific binding under the conditions of the assay. This selection is readily performed, and produces a superior display platform for the assay.

The second major innovation is the use of cells or viruses displaying antibodies or other molecular recognition elements by chemical, rather than genetic attachment. This approach allows the use of molecular recognition elements for which the encoding DNA sequence is not known, or which do not express well in the desired cell or virus. It allows the use of full-length antibodies instead of scFvs fragments, which may have poorer affinity. Chemical attachment can use, e.g., amine or carboxyl groups on the cell or virus, or a molecular recognition element which bonds to it. Of particular interest is the use of a genetically-displayed anchor such as a molecular recognition element (e.g., Protein A), or affinity tag (e.g., biotin or avidin) which supports the easy coupling of a variety of molecular recognition elements.

Third, we introduce the use of force-mediated stringency to further reduce non-specific background by removing non-specifically-bound DNA-containing reporters. Stringency force can be applied in various ways, including magnetic force for magnetic particles or cells or viruses decorated with magnetic particles, centrifugal force, and electrophoretic and dielectrophoretic forces, especially for virus particles and molecular conjugates of DNA and molecular recognition element.

Finally, rather than detecting all the bound DNA labels captured onto a surface or particle, either in situ or by non-specific elution, we use selective elution (particularly by competition) to free only DNA labels specifically bound by interaction with analyte for detection by PCR, further reducing the impact of any non-specific binding.

Other types of assays have been pursued, though they have not achieved the broad utilization of the solid-phase binding assays. Of particular interest are homogeneous assays, in which binding (or the suppression of binding, or competition) gives rise to the presence or absence of a signal. Examples of this sort of assay include the assembly of functional enzymes from split domains, the appearance of fluorescence when certain dyes intercalate into double-stranded nucleic acids, and molecular beacons which become fluorescent after a conformational change induced by the presence of a hybridization partner nucleic acid strand.

Tracking of particles and labels (in one or many interrogation areas) is common, though not much used for assays of analytes. The well-known lateral-flow assay involves the capture of particulate and/or enzymatic labels at pre-selected locations when analyte is present to bridge them to capture antibodies. Particle tracking is widely performed in 2 and 3 dimensions for velocimetry; particle image velocimetry (derived from laser speckle velocimetry) also is widely used for velocimetry. These methods can use a wide variety of methods of illumination and imaging, some of which are listed in Table 1. Of particular importance are time-varying, strobed, and sheet illumination, and observation by fluorescence and light scattering. Particle motion and tracking can also be used to characterize particles themselves, as in dynamic light scattering and in the nanoparticle tracking analysis practiced by Nanosight, Inc.

A specific application of tracking in the present invention is the magnified-imaging tracking of organisms coupled with computational analysis of their (non-Brownian) motion. Motile organisms suitable for magnified-imaging tracking include bacteria, protozoa, parasites, and crustaceans such as Daphnia, and they optionally can be stained (especially with fluors) to make tracking them easier. The distinctive motion of motile organisms can facilitate their detection, and can be used to detect their intoxication by substances in the liquid around them.

A variety of computational methods are known for extracting the motional behavior of particles in a suspension, and these can be applied in the present invention. For example, particle tracking is a common technique used to track the motions of colloids and nanoparticles in fluid suspensions. In this technique, the positions of all of the particles are located by identifying the local maxima in the intensity of a brightfield or fluorescence micrograph, and then refined to obtained sub-pixel accuracy by calculating a weighted center-of-mass of the region around each local maximum. To eliminate spurious features, features that exhibit shapes or sizes that are not characteristic of the particles of interest are eliminated. These techniques can be generalized to locate the positions of nonspherical particles. The positions of particles are then linked into trajectories by minimizing the total displacement of features between consecutive images in a time series. From these trajectories, metrics calculated and used to characterize motility can include: (a) the speed of a particle, calculated from the displacement of the particle as a function of time; (b) the persistence length or distance over which the particle moves in a straight line, calculated from the correlation function of the cosine of the angle between velocity vectors as a function of time; (c) the turning radius, calculated from the radius of curvature of the trajectory; (d) the mean-square displacement of a particle, calculated directly from the trajectory as a function of time and used to identify particles that are moving faster than diffusion.

Other methods based on standard high-throughput image processing techniques are also applied to identify changes in the motion of particles: (a) In motion vector analysis, the cross-correlation between corresponding regions (e.g. rectangles) in consecutive images in a time series is used to determine the displacement vectors of specific regions in the sample. In the present invention, coordinated motion towards an attractant or stimulant can be automatically detected from correlating the displacement vectors of specific regions. (b) In differential dynamic microscopy, the intensity fluctuations between images in a time series are calculated by computing the average difference in intensity for each pixel. Changes in average motion in response to a stimulant can be automatically detected from the growth of intensity fluctuations as a function of time.

### BRIEF SUMMARY OF THE INVENTION

The present invention provides a methodology for bioassays and diagnostics in which a particulate label (ranging in size from nm-scale molecular assemblages to organisms on the scale of tens or hundreds of microns), such as, but not limited to, nanoparticles, bacteria, bacteriophage, Daphnia, and magnetic particles, serve as labels or carriers for analytes bound by molecular recognition elements such as antibodies, aptamers, etc. Both particle types and detection methods are further listed in Table 1. The described methodology is generally applicable to most pathogen assays and molecular diagnostics. The present invention also leads to enhanced sensitivity and convenience of use.

The methodology in one aspect includes a method of assaying an analyte including at least the steps of: contacting the analyte with a plurality of viruses or cells, said viruses or cells each containing multiple copies of a DNA or RNA sequence capable of detection by means comprising nucleic acid hybridization or amplification, said viruses or cells also being capable of binding with the analyte; separating viruses or cells which have bound with the analyte from viruses or cells which have not bound with the analyte; detecting the multiple copies of a DNA or RNA sequence capable of detection by means comprising nucleic acid hybridization or amplification, by means comprising nucleic acid hybridization or amplification; and using the presence of the DNA or RNA sequence to infer the presence or concentration of the analyte.

The methodology in one aspect includes a method of detecting a motile organism in a sample, comprising the steps of: including at least the steps of: optionally pre-treating the sample to remove particulates larger than the organism, convert the organism into a more-motile form, or stain the organism to enhance detectability; and observing the organism by digital imaging with magnification; using a computer program to analyze the imaging results to determine the presence of objects with non-Brownian motility in the sample, and using the presence of objects with non-Brownian motility in the sample to determine the presence of the motile organism.

The methodology in one aspect includes a method of detecting intoxication of a motile organism by a sample comprising the steps of: contacting the organism with the sample, observing the motion of the organism by digital imaging with magnification; using a computer program to analyze the imaging results to characterize the motility of the organism in the sample, and using changes in the motility of the organism to determine whether it is intoxicated by the sample.

The methodology in one aspect includes a method of assaying an analyte comprising the steps of contacting the analyte with an interior wall of a channel or pore across which an electrostatic potential difference is maintained, said wall being associated with a molecular recognition agent capable of binding the analyte; and using changes in the current flowing through the pore or channel to infer the presence or concentration of the analyte.

The methodology in one aspect includes a method of assaying an analyte comprising the steps of contacting the analyte with a particle, said particle being associated with a molecular recognition agent capable of binding the analyte, and passing the particle into a channel or pore across which an electrostatic potential difference is maintained, and using changes in the current flowing through the pore or channel to infer the presence or concentration of the analyte.

The methodology in one aspect includes a method of assaying an analyte comprising the steps of contacting the analyte with an interior wall of a channel inside a vibrating structure, said wall being associated with a molecular recognition agent capable of binding the analyte; and using changes in the vibration of the vibrating structure to infer the presence or concentration of the analyte.

The methodology in one aspect includes a method of assaying an analyte comprising the steps of contacting the analyte with a particle, said particle being associated with a molecular recognition agent capable of binding the analyte; passing the particle into a channel inside a vibrating structure; and using changes in the vibration of the vibrating structure to infer the presence or concentration of the analyte.

The methodology in one aspect includes a method of assaying an analyte comprising the steps of contacting the analyte with an antibody capable of binding to the analyte; optionally separating antibody bound to the analyte from antibody not bound to analyte, using means selected from the group consisting of washing, fluid flow force, electrophoretic force, buoyant force, magnetic force, centrifugal force, or dielectric force; contacting the antibody with a moiety comprising an aptamer capable of binding to the antibody, and optionally also comprising non-aptamer DNA or RNA; optionally separating the moieties comprising an aptamer which are bound to the antibody, from moieties comprising an aptamer, not bound to antibody, using means selected from the group consisting of enzymatic digestion, washing, fluid flow force, electrophoretic force, buoyant force, magnetic force, centrifugal force, or dielectric force; optionally separating DNA or RNA contained in moieties comprising an aptamer, from other parts of the moiety using means selected from the group consisting of heat, denaturants, redox agents, competitors, enzymatic digestion, washing, fluid flow force, electrophoretic force, buoyant force, magnetic force, centrifugal force, or dielectric force; detecting the aptamer, DNA or RNA, and using the presence of the DNA or RNA to infer the presence or concentration of the analyte.

The methodology in one aspect includes a method of assaying an analyte comprising the steps of contacting the analyte with a plurality of particles, said particles each containing multiple copies of a single DNA or RNA sequence, said particles also being capable of binding with the analyte; separating particles which have bound with the analyte from particles which have not bound with the analyte using means selected from the group consisting of fluid flow force, electrophoretic force, buoyant force, magnetic force, centrifugal force, or dielectric force; detecting the multiple copies of the DNA or RNA sequence moieties, and using the presence of the DNA or RNA sequence to infer the presence or concentration of the analyte.

The methodology in one aspect includes a method of assaying an analyte comprising the steps of contacting the analyte with a surface having a plurality of separated sources of or passages for light, said surface also being associated with a molecular recognition agent capable of binding with the analyte; optionally separating analyte not bound to the surface from analyte which has bound to the surface; optionally staining or darkening the analyte by chemical reaction or binding of a darkening moiety; observing the intensity of individual sources of light; and using the darkening of the separated sources of or passages for light to infer the presence or concentration of the analyte.

### BRIEF DESCRIPTION OF THE FIGURES

FIG. 1. Using multi-copy quantitative PCR (qPCR) amplification as the read-out for the detection of micro RNA:DNA hybrids with immuno-phage particles.

FIG. 2. Schematic illustrating our approach to generating concatemers consisting of multiple self-complementary primer binding sites and inter-primer regions.

FIG. 3. Real-time quantitative PCR of two templates.

FIG. 4. Map of the RNA:DNA scFv expressing phagemid pIT2.

FIG. 5. Immobilization of biotinylated single stranded DNA oligonucleotides on the surface of magnetic streptavidin coated beads.

FIG. 6. Immobilization of thiolated single stranded DNA oligonucleotides on a DSP/PEG treated surface.

FIG. 7. Antigen detection using phage-displayed single-chain antibody fragments.

FIG. 8. Use of DNA coated magnetic beads to sequester micro RNA from solution for phage-depletion assay.

FIG. 9. An Immuno-magnetic Urchin Particle.

FIG. 10. Concentration dependent detection of immuno-magnetic urchins using real-time PCR.

FIG. 11. Concentration dependent detection of micro RNA mimics hybridized to their complementary DNA using urchin immuno-magnetic particles.

FIG. 12. A mixed monolayer of discrete-length poly(ethylene) glycol (PEG) molecules is used to inhibit non-specific biomolecule adsorption onto the surface and to provide a flexible linker to capture ligands.

FIG. 13. Multiplex detection of proteins and micro RNA using urchin immuno-magnetic particles.

FIG. 14. Multiplex detection of four marker proteins and three marker miRNAs in nine wells of a qPCR plate.

### DETAILED DESCRIPTION OF EMBODIMENTS

The present invention provides a methodology for bioassays and molecular diagnostics in which specific molecules and/or biomarkers are detected or quantitated through the effect their presence exerts on the location, properties, or behavior of particulate reporters. The particle can report the presence of the analyte by changes in its location, by changes in its properties such as mobility, density or optical properties, or it can comprise detectable moieties whose detection is modulated by the presence of the analyte. In some embodiments the particulate label is the analyte itself, or a form, derivative or portion thereof.

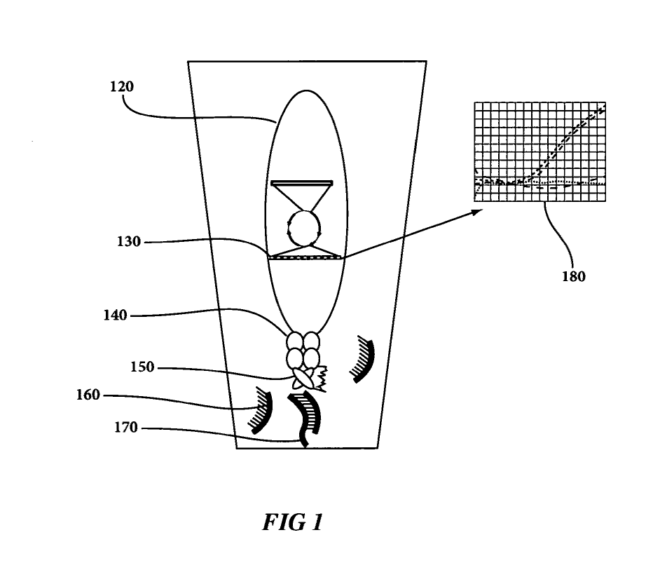

FIG. 1 is an example of using multi-copy quantitative polymerase chain reaction (qPCR) amplification as the read-out for the detection of micro RNA:DNA hybrids with immuno-phage particles. PCR is a technique now common in molecular biology to amplify a single or a few copies of a piece of DNA across several orders of magnitude, thereby generating thousands to millions of copies of a particular DNA sequence. An M13 bacteriophage 120 is shown. A single-chain antibody fragment 150 (scFv), encoded by a recombinant phagemid, is expressed as a fusion to the phage's capsid protein III 140. The antibody 150 recognizes micro RNA 160 hybridized to surface immobilized complementary DNA capture oligonucleotides 170. The multi-copy PCR template 130 is represented within the M13 phage 120. A quantitative qPCR readout 180 is obtained.

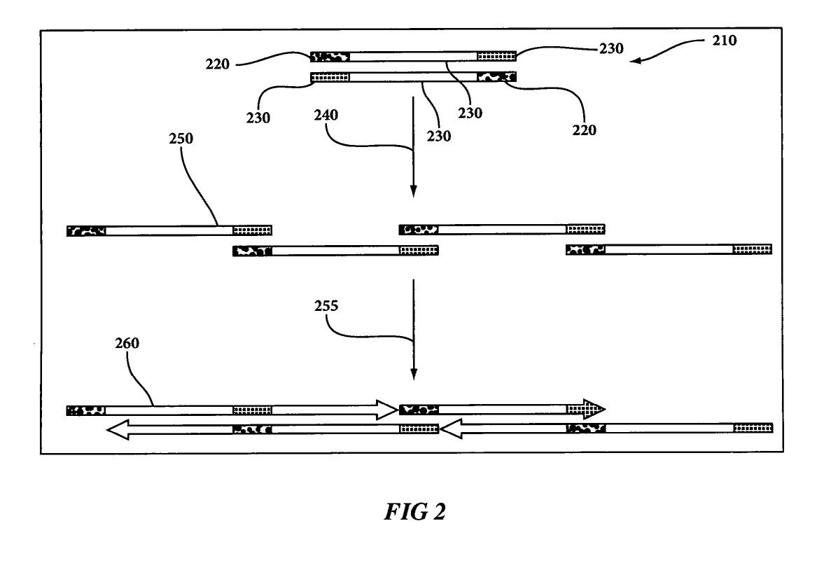

FIG. 2 is a schematic illustrating our approach to generating concatemers. Concatemers are long continuous DNA molecules that contain multiple copies of the same DNA sequences in series. Beginning with a PCR template 210 consisting of complementary primer binding sites 220, 230 and inter primer regions 230; in a first stage of denaturation and slow annealing 240 the moieties align and in a second stage 250 of polymerization the concatemers are completely generated.

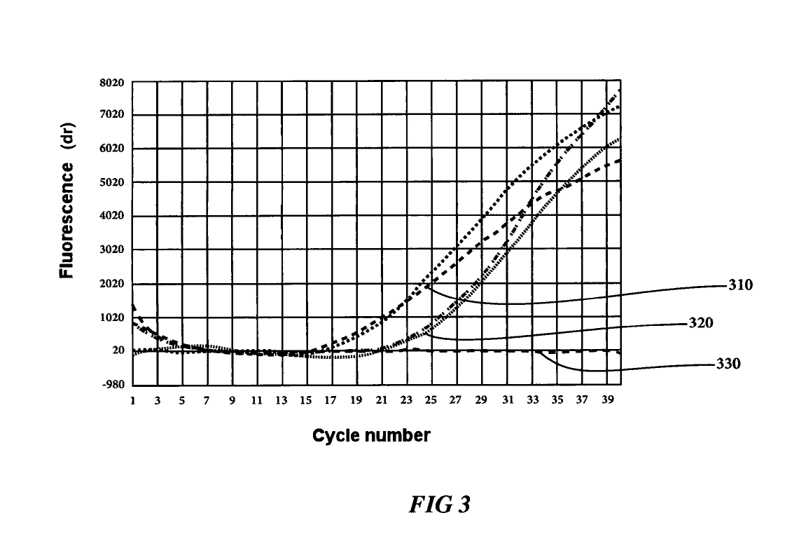

FIG. 3 illustrates a real-time quantitative PCR of two templates, one 320 containing 1 copy per template of a 75 bp PCR DNA fragment, and one 310 containing 14 copies per template. Both templates were analyzed in duplicate. A no-template control 330, containing just the primer and the reaction mix is shown as well.

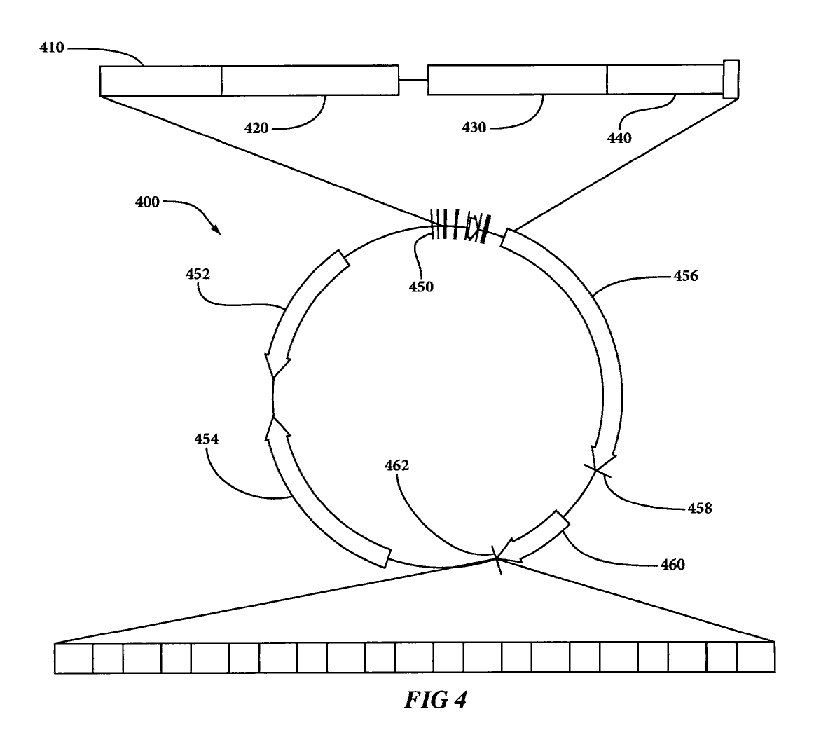

FIG. 4 is a map of the RNA:DNA single-chain variable fragment (scFv) expressing phagemid pIT2, indicating the proposed insertion site for our multi-copy PCR template. peIB 410: leader peptide VH 420: variable heavy chain VL 430: variable light chain gpIII 440: M13 protein III.

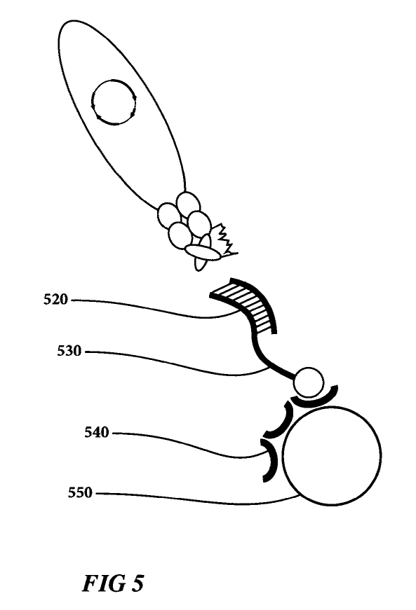

FIG. 5 demonstrates Immobilization of biotinylated single stranded DNA oligonucleotides 530 on the surface of magnetic streptavidin coated 540 beads 550. A RNA:DNA hybrid 520 is recognized by a phage-expressed single-chain antibody.

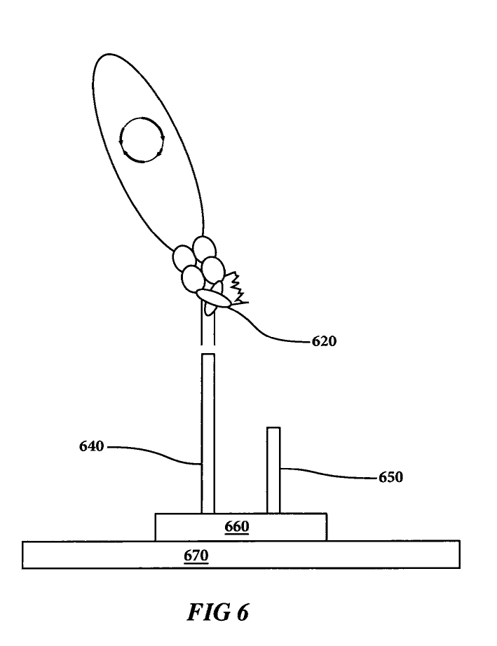

FIG. 6 demonstrates Immobilization of thiolated single stranded DNA oligonucleotides on a DSP/PEG (polyethylene glycol) 660 treated goldcoated surface 670. A RNA:DNA hybrid 620 is recognized by a phage 610 expressed single-chain antibody. A long chain PEG with maleimide 640 is represented along with a short chain PEG 650.

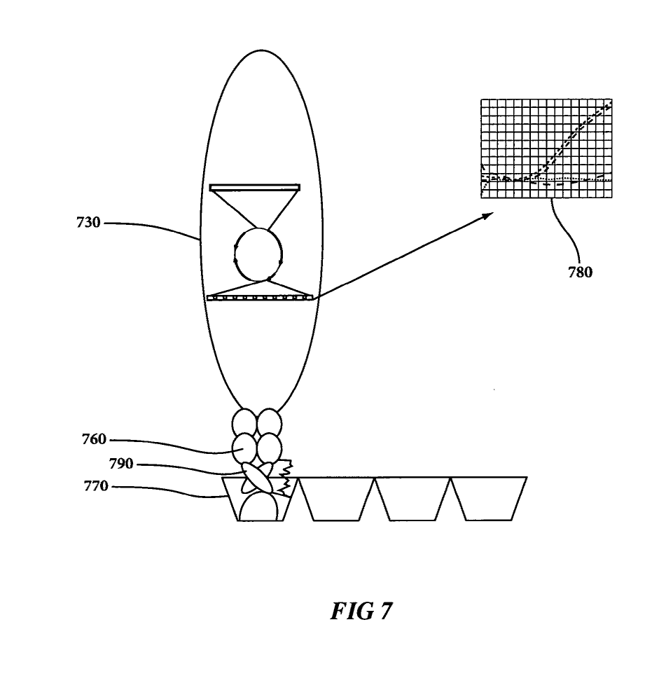

FIG. 7 represents antigen detection using phage-displayed single-chain antibody fragments. M13 phages 730 bound to the antigen 770 are lysed in situ and their nucleic acid is amplified by quantitative real-time PCR 780. 760 represents the pill protein and 770a short chain variable fragment (scFv).

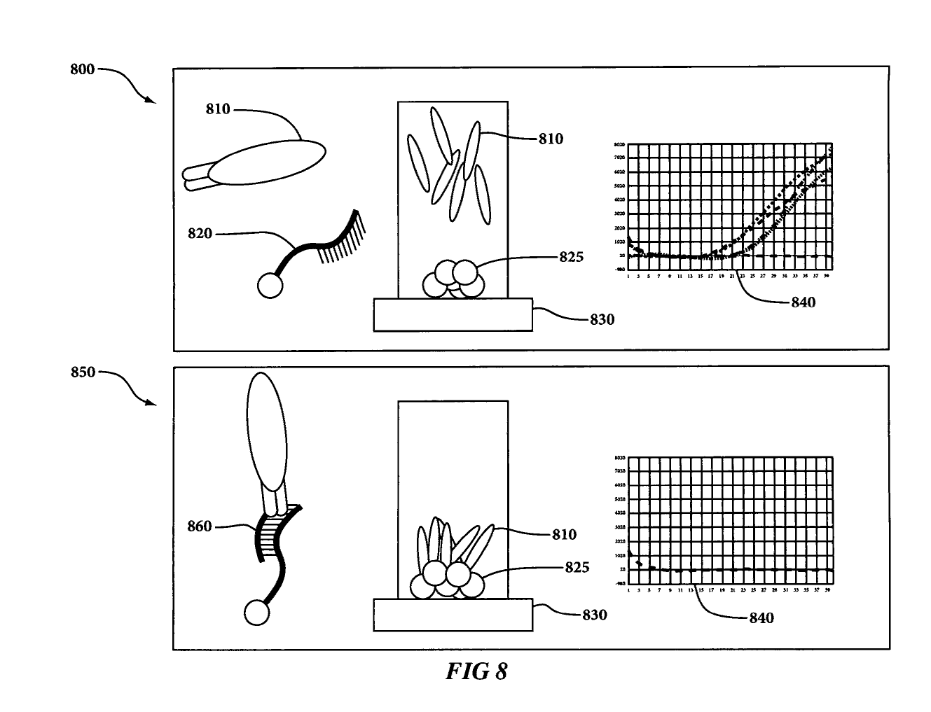

In FIG. 8 DNA coated magnetic beads 820 are used to sequester micro RNA from solution for phage-depletion assay. In the top panel in the absence of the targeted micro RNA, immuno-phages 810 do not bind the beads 825, which, after exposure to an external magnet 830, accumulate at the bottom of the reaction compartment. Phages 810 are free in the supernatant and will yield a strong signal when used as templates for quantitative PCR (qPCR) 840. In the bottom panel the presence of micro RNA 860 in the sample leads to the formation of the antigen (RNA:DNA hybrid) which is recognized by the immuno-phage particles. The phages 810 are subsequently bound to the magnetic beads 825. Fewer phage particles in the supernatant will lead to a weaker signal in the qPCR.

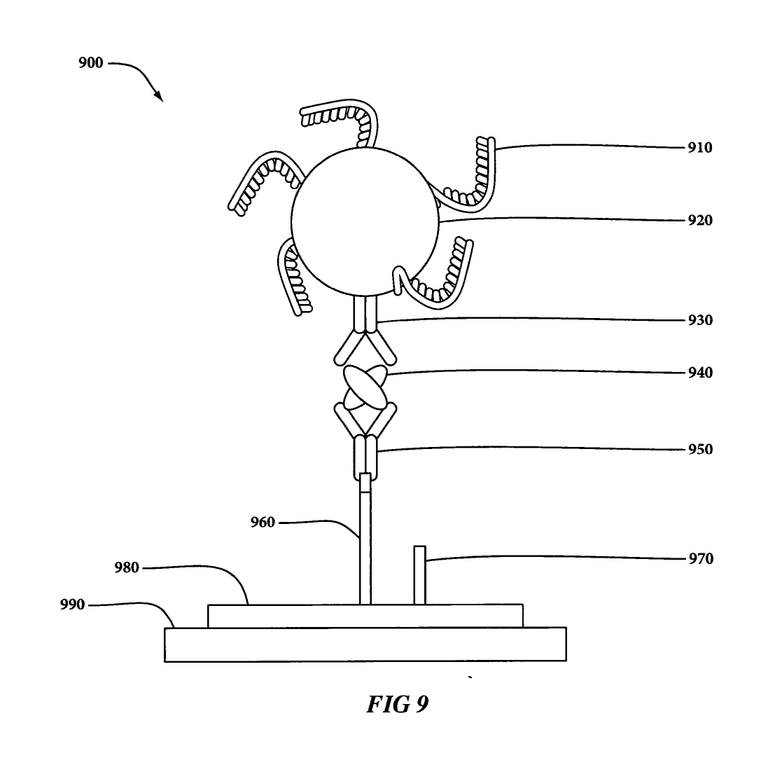

FIG. 9 demonstrates an Immuno-magnetic Urchin Particle. A magnetic particle 920 with attached DNA labels 910 is attached via a monoclonal VEGF antibody 930 to a VEGF protein 940 and a polyclonal VEGF antibody 950. These are attached to a gold coated surface 990 coated with DSP 980 and short 970 and long chain 960 PEG molecules with maleimide. These particles can be captured by single analyte molecules, and will subsequently be detected by qPCR. Specificity is enhanced by magnetic pull-off stringency. VEGF is shown as a sample protein.

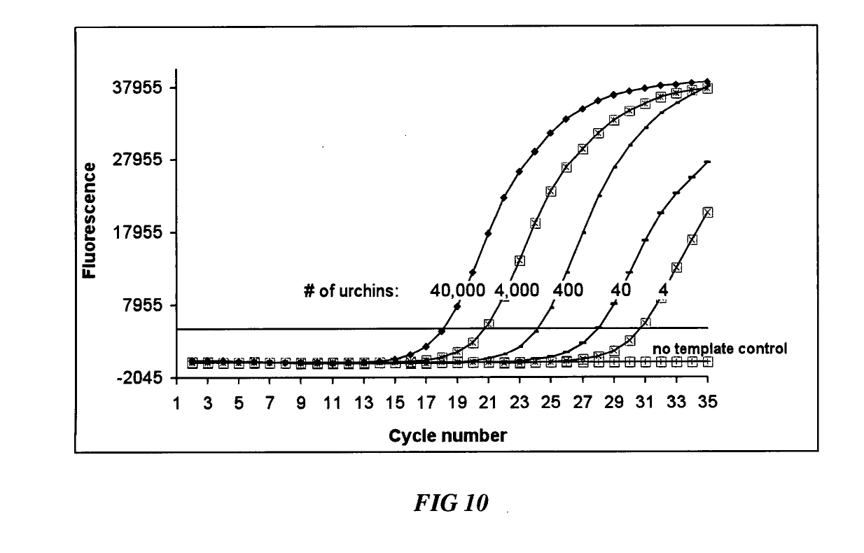

FIG. 10 shows the results of concentration dependent detection of immuno-magnetic urchins using real-time PCR. A solution of 250 nm magnetic particles carrying both the anti-RNA/DNA antibody, S9.6, and DNA at a ratio of 1:500 was serially diluted and amplified by standard qPCR.

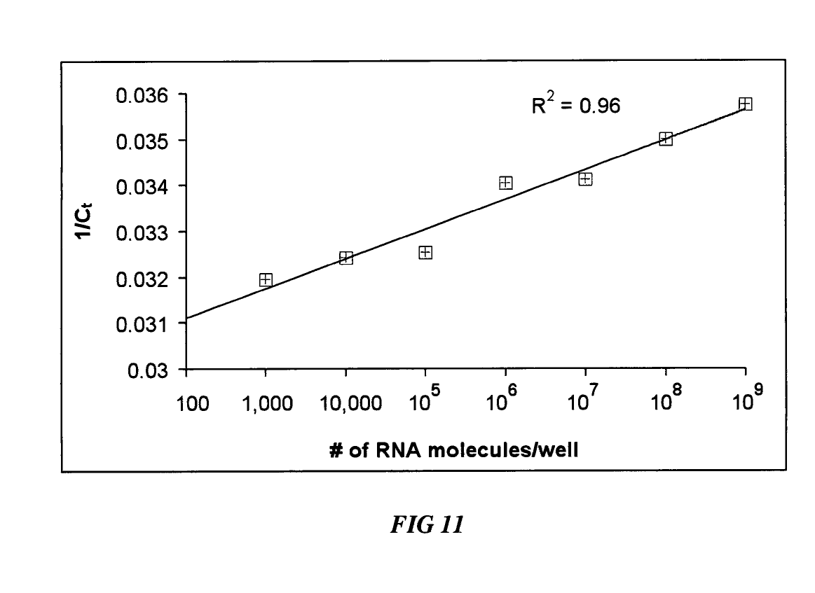

FIG. 11 exhibits an example of the concentration dependent detection of micro RNA mimics hybridized to their complementary DNA using urchin immuno-magnetic particles. Ct: Detection threshold in qPCR. <100 copies gave no amplification

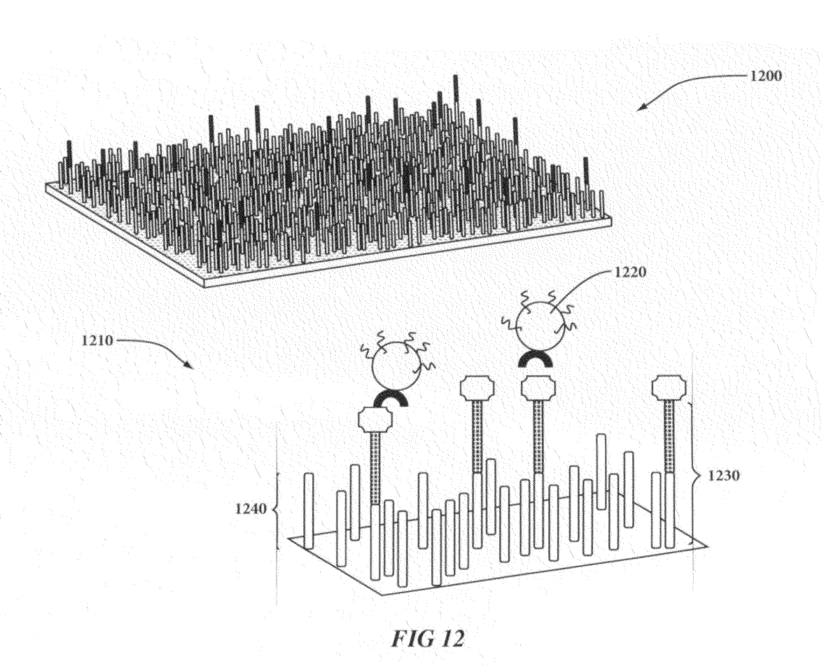

FIG. 12 shows a mixed monolayer of discrete-length poly(ethylene) glycol (PEG) molecules (short capped PEG molecules 1240 and long PEG molecules 1230) is used to inhibit non-specific biomolecule adsorption onto the surface and to provide a flexible linker to capture ligands and capture immunomagnetic urchin particles 1220.

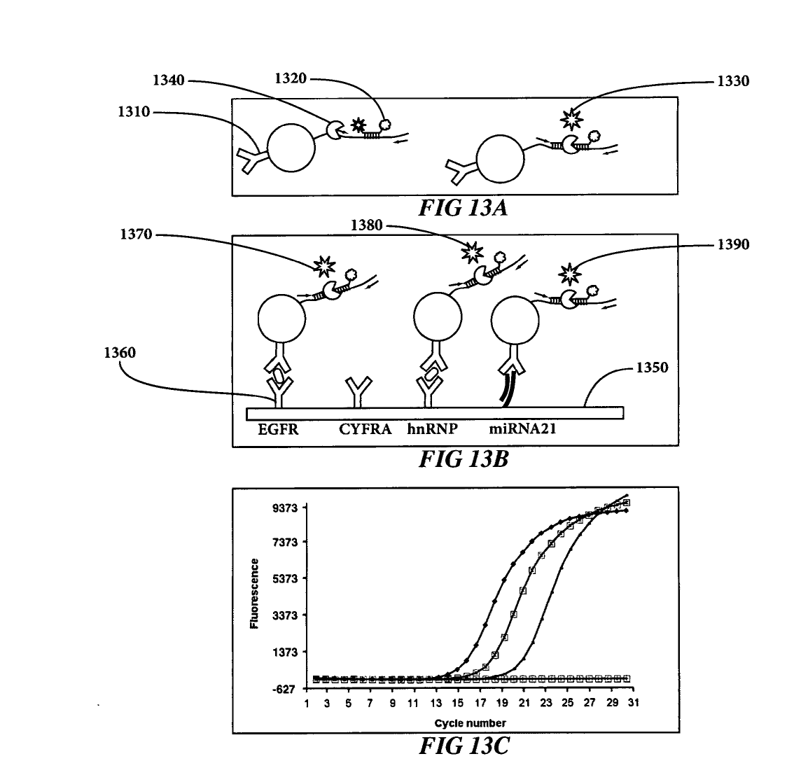

FIG. 13 illustrates multiplex detection of proteins and micro RNA using urchin immuno-magnetic particles. In 13A particles are decorated with antibodies 1310 and a DNA reporter sequence 1320; complementary to a 5′-nuclease probe (red). Each probe contains a unique fluorophore, proximal to a specific quencher molecule. Prior to polymerization Taq polymerase 1340 hydrolyzes the probe and the now fluorescent dye 1330 is released. In 13B multiple different antibodies 1360 are immobilized on the surface of a qPCR plate 1350, each recognizing a different target protein. Binding of the target subsequently retains the urchins, each carrying a unique fluorophore (1370,1380,1390). Micro RNA hybridizes to a complementary DNA sequence immobilized on the surface, and is recognized by urchins containing a unique RNA:DNA specific antibody. Then as shown in 13C during real-time PCR the fluorescent signal from multiple different urchins is recorded simultaneously.

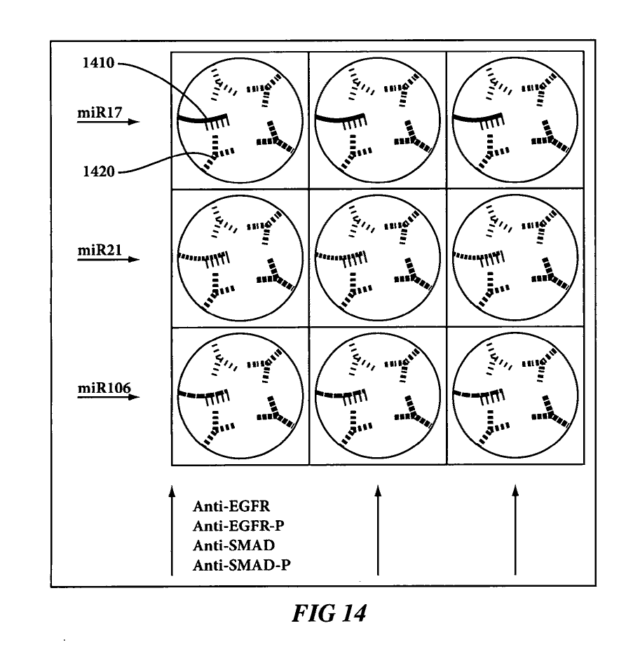

FIG. 14 illustrates multiplex detection of four marker proteins and three marker miRNAs in nine wells of a qPCR plate. Each well will be coated with antibodies 1420 against the target proteins, plus a single-stranded DNA molecule 1410 to capture a specific miRNA target. Equal volumes of samples and a mixture of target specific urchins are added to each well, and the reaction is monitored in a real-time PCR instrument. Targets are identified by the unique fluorophore on their specific urchin, and the position on the microtiter plate.

In accordance with an embodiment of this invention, a specimen is optionally pre-treated for concentration of the analyte, removal of particulates, contaminants or reaction inhibitors, reduction of viscosity, improvement of handling properties, or to modify the analyte for improved detection.

In an embodiment, the analyte of interest is a pathogen, virus, cell, DNA, mRNA or miRNA, a mutated and modified (e.g., methylated) DNA, an RNA splice variant, a protein, a peptide, a hormone, a biomarker, a toxin, or a modified (e.g., phosphorylated or acetylated) protein.

In an embodiment, the specimen in which the analyte is to be detected comprises a biopsy specimen, blood, serum, plasma, stool, saliva, sputum, cerebrospinal fluid (CSF), lavage fluid, nasal wash, urine, cell lysate, circulating tumor cells, fine needle aspiration biopsy (FNAB) cells, fluorescence-activated cell sorting (FACS) fraction, immunomagnetic isolate, air filtrate, formalin-fixed, paraffin-embedded (FFPE) slices, fresh-frozen specimens, drinking water, natural water, sea water, soil water, soil leachate, fresh tissue, frozen tissue, neutral formalin-treated tissue, formalin fixed paraffin embedded tissue block, or an ethanol-fixed paraffin-embedded tissue block.

In an embodiment, the readout method by which the analyte is detected is by determination of the presence or absence of the label in locations different from the locations expected in the absence of the analyte.

Accordingly, in an embodiment, the presence of the label is detected by means comprising light scattering, opacity, fluorescence, imaging, microscopy, chromatography, mass spectrometry, autoradiography, scintillation counting, or electrophoresis.

Accordingly, in an embodiment, the readout method by which the analyte is detected comprises the occurrence of a chemical or biochemical reaction dependent on the presence of a component of the label.

Accordingly, in an embodiment, the readout method by which the analyte is detected comprises polymerase- or helicase- or ligase-dependent amplification of nucleic acid sequences associated with the label, where the label comprises a virus, phage, cell protein or particle associated with a nucleic acid.

Accordingly, in an embodiment, the label comprises a virus, phage, cell, nanowire, protein or particle associated with a nucleic acid and also associated with a molecular recognition element such as an antibody, aptamer, nucleic acid, antibody analog, lectin, receptor, hormone, toxin, or drug or drug analog.

In an embodiment, the readout method by which the analyte is detected is modification of the density, optical properties, mobility, or buoyancy of the label to values different from the values expected in the absence of the analyte.

Accordingly, in an embodiment, the label is particle to which other particles are bridged in the presence of the analyte, and the readout method by which the analyte is detected is modification of the density and/or mass of the label by settling, flotation, field-flow fractionation or measurement of the buoyant mass of particles, e.g. though use of a vibrating sensor such as a cantilever containing a flow channel.

Accordingly, in an embodiment, the label is a particle which occupies volume to displace a competitive moiety (including without limitation, water, a dye, a fluor, or a conductor) from a location.

Accordingly, in an embodiment, the label is a particle of near neutral buoyancy which settles to form, with others, a packed bed of particles when its density is increased by analyte-responsive bridging to or deposition of a dense moiety to the particle.

Accordingly, in an embodiment, the label is a Brownian agarose particle which settles when bridged to gold nanoparticles by antibodies on the particle and a different type of antibodies on the nanoparticles, in the presence of an analyte capable of binding to both types of antibodies.

Accordingly, in an embodiment, the label is a 5 um fluorescently-labeled dextran particle bearing antibodies to a virus, where the virus is also recognized by a second antibody conjugated to 2 nm gold particles, and the particle can be centrifuged through water to stop at the interface with a sucrose solution, but only after silver deposition on the particle which occurs when the virus is present to bridge the second antibodies to its surface.

Accordingly, in an embodiment, the label is a buoyant microbubble of overall buoyant density 0.995 bearing antibodies to a virus, where the virus is also recognized by a second antibody conjugated to 2 nm gold particles, and the microcapsule can be centrifuged down through water to form a detectable and precipitate quantifiable by the height of a packed particle bed, but only after silver deposition on the bubble which occurs when the virus is present to bridge the second antibodies to its surface.

Accordingly, in an embodiment, the label is a 5 mm fluorescently-labeled polystyrene particle bearing antibodies to a virus, where the virus is also recognized by a second antibody conjugated to 150 nm paramagnetic particles, and the particle can be magnetically drawn into a 40 micron microfluidic passage with a 1 micron exit hole, to form a detectable packed bed of fluorescent particles, but only in the presence of the virus which can bridge the magnetic particles to the fluorescent particles to make the magnetically-responsive.

Accordingly, in an embodiment, the label is a CD4-positive white blood cell which can be recognized by an antibody conjugated to 150 nm paramagnetic particles, and the cell can be magnetically drawn into a 300 micron microfluidic passage with a 1 micron exit hole, to form a detectable packed bed of CD4 cells, where other cells can be discriminated against by their lesser magnetic mobility and visual appearance, and magnetic particles not bound to cells can be discriminated against by the visual appearance of the layer of particles, which differs from the appearance of the layer of cells.

In an embodiment, the label is a particle capable of nucleating bubble formation or freezing, conjugated to a molecular recognition element.

Accordingly, in an embodiment the label is a bacterial particle or protein capable of nucleating the freezing of subcooled water.

Accordingly, in an embodiment, the label comprises a concavity which tends to retain gas or vapor, from which bubbles can be nucleated in a supersaturated or superheated liquid.

In an embodiment, the readout method by which the analyte is detected is modification of the density, optical properties, mobility, or location of the label to values different from the values expected in the absence of the analyte.

Accordingly, in an embodiment, particles are increased in their tendency to adsorb to a surface, cantilever, observation area, rolling plane, cantilever, channel inside a cantilever, channel wall, Coulter principle counter channel wall, fiber, liquid interface, gas interface, particle, or pillar in the presence of the analyte.

Accordingly, in an embodiment, particles are reduced in their tendency to adsorb to a surface, cantilever, observation area, rolling plane, cantilever, channel inside a cantilever, channel wall, Coulter principle counter channel wall, fiber, liquid interface, gas interface, particle, or pillar in the presence of the analyte.

Accordingly, in an embodiment, the labels are T7 phage displaying antibodies to, or chemically conjugated with aptamers to, a protein biomarker target, carrying 50 copies of sequences amplifiable with the same set of PCR primers.

In an embodiment, the labels are M13 phage displaying proteins capable of binding to murine IgG antibodies, carrying 200 copies of sequences amplifiable with the same set of PCR primers.

In an embodiment, the labels are M13 phage displaying biotin ligase-substrate peptides which have been biotinylated in vitro using biotin ligase, attached via streptavidin to biotinylated human IgG recognizing VEGF.

In an embodiment, the labels are phage displaying antibodies to a prion protein aggregates, also encoding luciferase, detected by a luminometer during growth in E. coli.

In an embodiment, the labels are M13 phage particles selected for low non-specific binding to unrelated polyclonal rabbit antibodies bound by adsorption to a polystyrene microtiter plate, said phage being further engineered to display single-chain antibodies capable of binding to Junin virus particles, and carrying 20 copies of a single DNA sequence as PCR labels.

In an embodiment, the labels are phage displaying antibodies specific to RNA/DNA hybrids and also encoding luciferase. A DNA capture probe which has been exposed to a human serum sample containing miRNA of sequence complimentary to the capture probe is then exposed to the phage and washed. Bound phage are eluted with glycine pH 2.5 buffer and detected by a luminometer during growth in E. coli.

In an embodiment, the labels are phage selected for resistance to low pH, genetically modified to display antibodies to a protein biomarker target, detected by elution from surface-bound target and counted by use of a Spiral Plater.

In an embodiment, the labels are phage selected for resistance to low pH, genetically modified to display sequences from a human interleukin, added to a serum sample, contacted with an antibody to that interleukin. Phage not bound to the antibody because of competition by the hormone in the sample are counted by use of a Spiral Plater.

In an embodiment, the labels are phage whose coat proteins display peptides with affinity for cadmium selenide (CdSe) quantum dots, made fluorescent by binding of quantum dots, and whose tail proteins display antibody scFv (single-chain variable fragment) fragments with affinity to a bacterial pathogen, useful in detecting the pathogen.

In an embodiment, the labels are phage made conductive by the addition of gold nanoparticles conjugated to anti-phage antibodies, and then enhanced by deposition of silver enhanced by the gold nanoparticles, which are detected by their conductivity.

In an embodiment, the labels are non-phage viruses made fluorescent by binding an intercalating dye to the RNA contained within, and whose tail proteins display antibody scFv fragments with affinity to a bacterial pathogen, useful in detecting the pathogen.

In an embodiment, the labels are bacterial minicells made fluorescent by binding a fluorescent dye to the proteins contained within, and whose surfaces display antibody scFv fragments with affinity to a bacterial pathogen, useful in detecting the pathogen.

In an embodiment, the labels are magnetic nanoparticles carrying antibodies to a toxin, and also carrying 30 copies of a DNA sequence that is amplifiable by LAMP. The particles are contacted with a surface bearing anti-toxin antibodies loaded with the toxin. Magnetic pulloff is used to reduce non-specific binding, then particles are eluted and the tags quantified by qPCR.

In an embodiment, the labels are magnetic nanoparticles carrying antibodies to a toxin, and also carrying 30 copies of a DNA sequence that is amplifiable by LAMP. The particles are contacted with a surface bearing anti-toxin antibodies loaded with the toxin. Electrophoretic pulloff is used to reduce non-specific binding, then particles are eluted and the tags quantified by qPCR.

In an embodiment, the labels are magnetic nanoparticles carrying antibodies to a toxin, and also carrying 30 copies of a DNA sequence that is amplifiable by LAMP. The particles are contacted with a surface bearing anti-toxin antibodies loaded with the toxin. Centrifugal roll-off by applying a force parallel to the surface is used to reduce non-specific binding, then particles are eluted and the tags quantified by qPCR.

In an embodiment, the labels are magnetic nanoparticles carrying antibodies to a toxin, and also carrying 30000 copies of a DNA sequence that is amplifiable by LAMP attached using layer-by-layer assembly using polylysine. The particles are contacted with a surface bearing anti-toxin antibodies loaded with the toxin. Centrifugal roll-off by applying a force parallel to the surface is used to reduce non-specific binding, then particles are eluted, DNA freed by protease, and the tags quantified by qPCR.

In an embodiment, the labels are magnetic nanoparticles carrying antibodies to a toxin, and also carrying 30 copies of horseradish peroxidase are bound to immobilized samples, some of which contain toxin. Magnetic pulloff is used to reduce non-specific binding, then particles are eluted and the particles quantified by enzyme activity.

In an embodiment, the labels are magnetic nanoparticles carrying antibodies to a phosphorylated biomarker protein, and also carrying 30 copies of horseradish peroxidase are bound to immobilized samples, some of which contain phosphorylated biomarker. Magnetic pulloff is used to reduce non-specific binding, then particles are eluted and the particles quantified by enzyme activity.

In an embodiment, the labels are magnetic nanoparticles carrying PNA Peptide Nucleic Acid) probe sequences to a pathogen's 16 S rRNA, and also carrying 3000 copies of a DNA sequence that is amplifiable by LAMP. Immobilized total RNA from a sample is probed with the nanoparticles and washed. Magnetic pulloff is used to reduce non-specific binding, then particles are eluted and the tags quantified by qPCR.

In an embodiment, the labels are magnetic nanoparticles carrying PNA Peptide Nucleic Acid) probe sequences to a pathogen's 16 S rRNA, and also carrying 3000 copies of a DNA sequence that is amplifiable by LAMP, treated with 0.05 mM spermine to reduce charge density. Immobilized total RNA from a sample is probed with the nanoparticles and washed. Magnetic pulloff is used to reduce non-specific binding, then particles are eluted and the tags quantified by qPCR.

In an embodiment, an array of light sources smaller than 150 μm in diameter, such as LEDs, fibers, mirrors or orifices, decorated with antibodies to a pathogen. A serum sample potentially containing the pathogen is contacted with the surface, followed by washing. Binding of the pathogen to the antibodies produces a reduction in the brightness of at least three of the lights as seen by a CCD camera, and is interpreted as evidence of the presence of the pathogen.

In an embodiment, an array of light sources smaller than 50 μm in diameter, such as LEDs, fibers, mirrors or orifices, is decorated with antibodies to a pathogen. A serum sample potentially containing the pathogen is contacted with the surface, followed by washing, then a second antibody to the pathogen, where the second antibody is decorated with 15 nm gold nanoparticles. Chemical silver deposition around any nanoparticles bound produces a reduction in the brightness of at least 10 of the lights as seen by a CCD camera, and is interpreted as evidence of the presence of the pathogen.

In an embodiment, the labels are gold nanorods carrying PNA probe sequences to a pathogen's 16 S rRNA, and also carrying 3000 copies of a short PEG polymer which stabilizes them. Immobilized total RNA from a sample is probed with the nanorods and washed. Microwave irradiation is used to generate heat if the sample contains the pathogen RNA, which is detected by IR microscopy or liquid crystals, or release of a material from a fusible container.

In an embodiment, the labels are gold nanorods carrying PNA probe sequences to a pathogen's 16 S rRNA, and also carrying 3000 copies of a short PEG polymer which stabilizes them. Immobilized total RNA from a sample is probed with the nanorods and washed. Microwave irradiation is used to generate heat if the sample contains the pathogen RNA, which is detected by a thermopile.

In an embodiment, the labels are gold-clad magnetic nanoparticles carrying PNA probe sequences to a pathogen's 16 S rRNA, and also carrying 3000 copies of a short PEG polymer which stabilizes them. Immobilized total RNA from a sample is spotted on a field effect transistor (FET), and hybridized with the nanoparticles. Magnetic force is used to remove non-specifically bound particles, and the presence of remaining particles is detected by the FET.

In an embodiment, the labels are nanocapsules containing sulfur hexafluoride (SF6) and carrying aptamers to murine IgGs which are contacted with an immobilized bacterial pathogen decorated with murine anti-pathogen IgGs. After washing, the nanocapsules are eluted, and the SF6 liberated and detected by electron capture as an indicator of the presence of the pathogen.

In an embodiment, the labels are magnetic polymeric nanoparticles carrying aptamers to a bacterial pathogen and also carrying absorbed chemical tag molecules detectable by ion mobility spectrometry which are thermally desorbed and detected by ion mobility spectrometry.

In an embodiment, the labels are nanoparticles bridged to a polymer by way of two sandwiching antibodies the antigen, and analyzed by Coulter analysis using a nanopore.

In an embodiment, the labels are nanoparticles bridged to a polymer by way of two sandwiching antibodies, one of which bears a photoactive group to which polymer is bridged after the antibody associates with its target, and analyzed by Coulter analysis using a nanopore.

In an embodiment, the labels are nanoparticles bridged to a polymer by way of two sandwiching antibodies, one of which bears a photoactive group to which polymer is bridged after the antibody associates with its target, and analyzed by light scattering.

In an embodiment, the labels are magnetic polymeric microparticles with internal-surface-reversed-phase properties carrying aptamers to a bacterial pathogen and also carrying absorbed GC'able chemical tag molecules which are eluted with an organic solvent and detected by gas chromatography.

In an embodiment, the labels are liposomes encapsulating ATP and bearing antibodies to a protein are mixed in a thin layer of liquid, in the presence of low levels of an ATP-consuming enzyme to scavenge traces of free ATP, with firefly luciferase conjugated to near-IR-absorbing nanoshells also bearing antibodies to the same protein, and a blood serum sample. If the protein of interest is present in the sample it bridges the liposomes and the nanoshells. A bright flash of near-IR light is followed by luminometry; the presence of the protein is indicated by luciferase luminescence.

In an embodiment, the labels are liposomes encapsulating heme and bearing antibodies to a protein. Samples potentially containing the protein of interest are contacted with the surfaces of microwells bearing a different antibody to the protein. The wells are emptied and washed, and the heme-containing immunoliposomes are added, allowed to bind, and washed out. A small volume of high-pH solution is added to disrupt any bound liposomes, followed by a well-buffered solution of horseradish peroxidase apo-enzyme which can reconstitute with heme to form an active holo-enzyme, and chemiluminescence detection reagents. Detection of chemiluminescence by a charged coupled device (CCD)-based gel doc apparatus enables simultaneous measurement of multiple wells with high sensitivity.

In an embodiment, the labels are magnetic polymeric microparticles with internal-surface-reversed-phase properties carrying aptamers to a bacterial pathogen and also carrying absorbed chemical tag molecules which are eluted with an organic solvent and detected by gas chromatography or differential mobility analysis.

In an embodiment, the labels are aptamers to a viral target, fused to branched DNA molecules bearing multiple copies of sequences amplifiable by the same PCR primers.

In an embodiment, the labels are aptamers to a viral target, fused to branched DNA molecules bearing multiple copies of sequences amplifiable by the same PCR primers, treated with 0.2 mM spermidine in TE buffer.

In an embodiment, the labels are murine IgG1 antibody to a human hormone, bound by a DNA aptamer to the Fc region of murine IgG1 which is fused to branched DNA molecules bearing multiple copies of sequences amplifiable by the same PCR primers.

In an embodiment, the labels are murine IgG1 antibody to a human hormone, bound by a DNA aptamer to the Fc region of murine IgG1 which is fused to branched DNA molecules bearing multiple copies of sequences not found in the human genome and optimized for Solexa sequencing.

In an embodiment, the labels are magnetic nanoparticles carrying antibodies to a toxin, and also carrying 30 copies of a DNA sequence that is removable from the particle by treatment with a restriction enzyme for detection by sequencing.

In an embodiment, the labels are magnetic nanoparticles carrying antibodies to a toxin, and also carrying 30 copies of a DNA sequence that is removable from the particle by treatment with a restriction enzyme for detection as polonies.

In an embodiment, the labels are magnetic dextran nanoparticles carrying antibodies to a toxin, and also carrying 30 copies of an RNA sequence that is removable from the particle by treatment with a dextranase enzyme for detection by nucleic acid sequence-based amplification (NASBA).

In an embodiment, the labels are dextran polymers conjugated with antibodies to a toxin, and also carrying 30 copies of an RNA sequence that is removable from the polymer by treatment with a dextranase enzyme for detection by nucleic acid sequence-based amplification (NASBA).

In an embodiment, the labels are magnetic dextran nanoparticles carrying antibodies to PSA, and also carrying 3000 copies of a fluor that is removable from the particle by treatment with a dextranase enzyme for detection by fluorometry.

In an embodiment, the labels are wax-sealed capsules containing ammonium perchlorate as the solid component of a lithergolic mixture, detected by melting the wax with a flash lamp and triggering the energetic lithergolic reaction which is detected with a CCD camera.

In an embodiment, the labels are magnetic agarose nanoparticles carrying antibodies to dioxin, and also carrying 3000 copies of a fluor that is removable from the particle by treatment with an agarase enzyme for detection by fluorometry.

In an embodiment, the labels are microbial nanowires which are conjugated to a Fab′ of an antibody to ricin toxin, and captured by sandwich binding with ricin toxin onto a magnetic nanoparticle. The particles are washed and concentrated, then the nanowires are eluted and electrophoresed against a porous membrane decorated with interdigitating electrodes. The conductivity between the electrodes is measured, and elevated conductivity interpreted as indicating the presence of the toxin in the original sample.

In an embodiment, the labels are slightly-negative mutant silk protein polymer chains which are conjugated to a Fab′ of an antibody to ricin toxin, and captured by sandwich binding with ricin toxin onto a magnetic nanoparticle. The particles are washed and concentrated, then the silk chains are eluted and electrophoresed against a porous membrane decorated with interdigitating electrodes. Gold nanoparticles conjugated to anti-silk protein antibodies are added, the excess washed away, and silver salt and reductant added to deposit silver around the gold nanoparticles. The conductivity between the electrodes is measured, and elevated conductivity interpreted as indicating the presence of the toxin in the original sample.

In an embodiment, labels are magnetic dextran nanoparticles carrying radioactive labeled antibodies to biomarkers which can be detected by scintillation counter or autoradiography.

In an embodiment, labels are magnetic dextran nanoparticles carrying radioactive labeled DNA complement to disease DNA markers which can be detected by scintillation counter or autoradiography.

### EXAMPLES

### Example 1

### Demonstrating the Utility of an Engineered Immuno-Phage Particle for the Ultrasensitive Detection of Micro RNA in Blood Serum

Micro RNAs, or miRNAs—a species of small non-coding RNA—are emerging as widely-important agents of post-transcriptional regulation of coding genes through mRNA decay and/or translational repression. The role of miRNAs in human disease is just being understood. Micro RNA networks have been proposed to represent one of the ‘hidden layers’ of regulation that integrates the transcriptome (the complete set of RNAs) of a cell with its proteome (the complete set of proteins). Micro RNA binds to the 3′UTR of a target mRNA through a 7 bp seed region on the 5′ end (base pairs 2-8), sequesters the target mRNA in the RISC(RNA Induced Silencing Complex) and mediates post-transcriptional gene silencing through deadenylation-mediated mRNA decay and/or translational suppression by mechanisms not yet fully elucidated. At the current time miRNAs have been associated with multiple aspects of development, differentiation, proliferation, apoptosis and virtually every important cellular process. There are currently more than 700 described human miRNAs in the miRBase with the potential for at least 1000 miRNAs based on sequence prediction. Very few miRNAs have been linked to specific target mRNAs, but for those that have been defined it appears that one miRNA may target anywhere from one to several hundred mRNAs, and also that one mRNA may be the target of several miRNAs. This is consistent with the concept that a specific miRNA may act to regulate the expression of a group of genes encompassing a functional pathway. The dysregulation of micro RNA expression has been documented in the vast majority of cancers and contributes to tumor initiation and progression, and there is a significant interest in either targeting micro RNAs or using them as prognostic and diagnostic targets, as well as therapeutic agents. Altered miRNA expression has revealed signatures of miRNA expression correlated with specific malignancies and even some prognostic groups. The finding that approximately 50 percent of the known human miRNAs are located at cancer-associated regions of the genome suggests that miRNAs play a role in the pathogenesis of various human cancers. Therefore, the potential range of applications of the proposed technology will include all new cancer patients as well as patients under treatment, as the technology can be used for diagnostic purposes, prognostic prediction, and minimal residual disease detection. It is estimated that in the US alone, 2 million tests will be performed annually which potentially could be better addressed using our platform.

In an example, a phagemid containing multiple repeats of a short, amplifiable reporter sequence is engineered to encode a well-characterized phage displayed RNA:DNA specific single-chain antibody. In one possible application of this affinity reagent, the resulting immuno-phage particle can be used for the detection of specific micro RNAs in blood serum. The application takes advantage of an RNA:DNA-directed antibody with unusual properties in that, similar to the enzyme RNaseH, it recognizes exclusively RNA:DNA hybrid molecules. It does not significantly bind to single-stranded nucleic acids, nor does it recognize double-stranded DNA or RNA. As shown in FIG. 3, the antibody detects up to 100 pmol/dot of the RNA:DNA hybrid, but none of the control molecules, such micro RNA or the single-stranded complementary DNA alone. The binding of the immuno-phage particle to the target is reported through the amplification of phage sequences using real-time PCR. For this purpose, a multi-cassette real-time PCR target, consisting of a series of up to 200 short DNA cassettes, each flanked by two opposing primer binding sites, is constructed here. It is used for the multi-label, ultra-sensitive diagnostic amplifications. The parallel amplification of multiple sequences of the same nature results in a significantly increased signal response. In order to generate the multi-cassette template, two self-complementary primers (5A: 5′ GCC CAG CCC ACC CCC AGC CC 3′, and 3A: 5′ GGG CTG GGG GTG GGC TGG GC) are used for the amplification of a 75 nt synthetic oligonucleotide (GCC CAG CCC ACC CCC AGC CCA AAA ATA AAA AAA TAA AAA ATA AAA ATA AAT AAA AGC CCA GCC CAC CCC CAG CCC). The PCR product is separated from the primers by agarose gel electrophoresis, isolated using a Qiagen DNA gel extraction kit, and then serves as the template for a subsequent, primer-free PCR reaction. The amplification product is subjected to 25 cycles of repetitive denaturation, slow renaturation (30° C./h), and polymerization. Since the primers had originally been chosen to be a) much more GC-rich (85%) then the GC-free inter-primer region, and b) self-complementary, we had anticipated the generation of template concatemers, with the annealing of the primers favored over the annealing of the inter-primer region (FIG. 4). In the primer-free polymerization, the 3′-ends of the partially overlapping fragments would then serve as the starting point of what would be nucleotide fill-in reactions. T4 DNA ligase is used as recommended by the manufacturer (NEB) to close any potential nicks at the end of this reaction. Finally, the reaction is ligated into pGEM-T-EZ (Promega), transformed into E. coli, and individual transformants are analyzed by DNA nucleotide sequencing using primers binding outside of the multiple-cloning site of pGEM-T-EZ. While this particular TA-cloning vector is not a phagemid, it has convenient restriction sites (e.g. EcoRI, NotI) that allow the recovery of the complete multi-template cassette from the plasmid and its ligation into phagemid vectors. DNA sequencing has revealed that we have obtained a variety of multi-template repeats cloned into pGEM-T-EZ, ranging from 1 to 14 copies. In an effort to evaluate the suitability of our multi-template cassette plasmids for real-time PCR, we have compared amplification results for one of them (pUS7039, bearing 14 copies of the label sequence) with those using a single-template plasmid (pUS7037). For our real-time quantitation experiment, Agilent's Brilliant II QPCR mix is used, containing the double-stranded DNA specific dye SYBR Green I. 25 ml reactions are set up in duplicate for the respective plasmids, each containing equal amounts of template and primers 5A and 3A, shown in the previous paragraph. The reaction is incubated in a thermocycler for 10 min at 95° C., and 40 cycles of 30 sec at 95° C., 1 min at 57° C. and 1 min at 72° C. As shown in FIG. 5, both plasmid templates generate a template. In one example, the average Ct value for the single template plasmid was 24.61 and for the 14-copy template it was 19.78.

Briefly, bacterial cells harboring a phagemid is infected with helper phage KM13. This phage provides all the necessary proteins necessary for packaging any DNA with an f1-origin of replication (such as our phagemid) into a phage coating. Since the scFv fragment is translationally fused to the phage capsid protein III, it is displayed at the end of the phage (FIG. 1). Infected bacterial colonies are identified by the pseudo-plaques that are formed in a lawn of bacteria suspended in soft agar. In solution, phage can be separated from bacteria by centrifugation, followed by PEG precipitation. 10¹⁰ to 10¹² phage are isolated from one liter of bacteria, as determined after titering the phage stock on uninfected host bacteria.

We use a phage expressing a single-chain antibody fragment to detect hybrids between surface immobilized single-stranded DNA (ssDNA) and micro RNA. Multiple approaches for the attachment of the DNA capture molecules to solid surfaces are conceivable, for instance, ssDNA oligonucleotides will be attached to individual wells of a microtiter plate, or ssDNA oligonucleotides will be attached to magnetic particles. At least two chemistries for the immobilization of the synthetic oligonucleotides to the solid surface of either a microtiter plate or paramagnetic beads are possible. In one option (FIG. 7) biotinylated oligonucleotides, offered by various oligonucleotide providers, are attached to the surface of streptavidin-coated magnetic beads, or streptavidin coated microtiter plates. In another option (FIG. 6), improved low-nonspecific-adsorption PEG chemistry is used. Amine-PEG and amine-PEG-Maleimide will be immobilized onto gold surfaces via a self assembled monolayer of Dithiobis-(succinimidyl)-propionate (DSP) deposited onto the surface prior to PEG immobilization. DSP contains an amine-reactive N-hydroxysuccinimide (NHS) ester at each end of an 8-carbon spacer arm. NHS esters will react with the primary amines of the PEG molecules to form a stable amide bonds. PEG with maleimide functionality at the other end from the amine is used to covalently immobilize thiolated DNA; here, p-Maleimidophenyl isocyanate could also be used as a heterobifunctional cross-linker to tether thiol-modified oligonucleotides to terminal OH groups on the PEG molecules. In another variation of the attachment method, we will use “DNA-BIND” plates (Corning, Cat. No. 2505), coated with N-oxysuccinimide esters which can also be coupled to free amines. A 9:1 ratio of amine-PEG and amine-PEG-Maleimide was found to give superior results in terms of reducing background binding.

RNA will be added to microtiter plates pre-coated with bait oligo-nucleotides, (or containing a suspension of bead-immobilized single-stranded DNAs, see below). The reaction is briefly heated to 95° C., and then cooled down to approximately 60° C. After 1 h at 60° C., the reaction is slowly (30° C./h) cooled down to 25° C. Excess RNA is removed through successive washes with PBS (pH 7.4). In the example (FIG. 9), a microtiter plate is coated with serial dilutions of a DNA oligonucleotide. After washing away unbound oligonucleotides, a 5-10 fold molar access of synthetic RNA, complementary to the DNA bait molecule, is added to each well. As a control, non-complementary RNA is added to some wells. In that case, we do not expect any hybrids to form. A specific numbers of phage particles is then added directly to each well (controls without phage are included throughout the experiment). After 30 min incubation at room temperature and three wash steps (with PBS, pH 7.4), specifically retained phage particles are lysed in situ, i.e. in the microtiter plate. The lysate is then used as a template for our multi-copy, parallel real-time PCR amplification.

Aside from following good laboratory practices (use of a hood, aerosol barrier tips, etc.), we have adapted the use of deoxyuracil (dUTP) and uracil-DNA glycosylase (UDG) to our parallel immuno-phage amplification method. The incubation of PCR reactions with UDG prior to thermal cycling in combination with the use of dUTP in the PCR amplification is a commonly used technology to prevent contamination. This nucleotide is generally well accepted by the polymerases used in PCR. The resulting DNA amplicons containing dUTP are susceptible to degradation by UDG, generic DNA molecules are not. The samples are incubated 15 min at 20° C. prior to the PCR reaction for UDG-mediated decontamination. This incubation is followed by standard qPCR, which includes a 10 min denaturation of the DNA, inactivating UDG.

### Example 2

### Detection of RNA:DNA Hybrid Molecules in Blood Serum without Pre-Purification

In one example, immuno-phage particles are used to detect micro RNAs in bovine serum spiked with various amounts of a synthetic specific micro RNA sequence. Magnetic beads coated with capture oligonucleotides are suspended directly in the serum sample, or various dilutions of a serum sample. In one approach, we keep the beads and the sample in suspension for 30 min to allow hybridization of any micro RNA to the capture DNA on the bead. In another approach we briefly heat the serum sample to 95° C., followed by centrifugation to remove any protein precipitate formed during the heating step. The DNA beads are then suspended in the supernatant as in the first approach. In both cases, defined numbers of immuno-phage particles are added to the bead suspension, and incubated for a short time, before a permanent magnet is used to collect the magnetic particles on the bottom of the reaction tube. The phage supernatant serves as a template for our quantitative PCR detection protocol, yielding a label-depletion assay for miRNA. FIG. 10 depicts the results.

### Example 3

### Detection of Specific Micro RNAs in Archived Chronic Lymphocytic Lymphoma (CLL) Samples

In one example, the technology is used for the detection of prognostic and diagnostic micro RNA markers for B-cell chronic lymphocytic leukemia. It is estimated that in the U.S. alone 1,437,000 new cancer cases (745,000 male and 692,000 female) occurred in 2008, including 15,000 cases of chronic lymphocytic leukemia. B-cell chronic lymphocytic leukemia (B-CLL) is the most common leukemia in the western world, but little is known regarding its initiation and progression. Patients with B-CLL follow a heterogeneous clinical course. Recently, micro RNA has emerged as an important tool for the diagnosis and prognosis of CLL.

New findings support the view that CLL is a genetic disease where the main alterations occur at the level of transcriptional/post-transcriptional regulation of the malignant cell's genome because of deregulations of micro RNAs. Specifically, miR-15a and miR-16-1, located at 13q14.3, are frequently deleted and/or down-regulated in patients with B-cell CLL. Both micro RNAs negatively regulate BcI2 at a post-transcriptional level and deletion of these micro RNAs can lead to decreased apoptosis in B-CLL. Thus, micro RNA analysis not only advances knowledge of pathogenesis of B-CLL at the microgenome level but also distinguishes normal B cells from malignant B cells in CLL. Additionally, a unique micro RNA signature has been reported to be associated with prognostic factors such as mutations in the immunoglobulin heavy-chain variable-region gene (IgV(H)) or high expression of the 70-kd zeta-associated protein (ZAP-70+) and disease progression in CLL. Furthermore, mutations in mmRNA transcripts are frequent, some of them germ-line, may have functional importance and may predispose to CLL and to a spectrum of associated malignancies. These findings suggest that micro RNAs will be important markers for the diagnosis and prognosis of B-CLL and other cancers.

Developing a versatile and parallel detection platform with improved analytical sensitivity and specificity for micro RNA cancer biomarkers is the major innovative goal of the proposed work.

By utilizing a phage-immuno PCR method the challenges of antibody/DNA conjugation are avoided while still allowing for the amplification afforded by PCR or similar isothermal amplifications.

In this example, the multiple-label amplification, immuno-phage technology is used for the analysis of clinical oncology samples. Specifically, the immuno-phage detection particles are used to probe cell lines for the presence of certain prognostic and diagnostic micro RNAs. Cell lines known to not express the targeted micro RNA serve as negative controls in such an assay. After making serial dilutions of the cells to be assayed into the control cells (1:10→1:10⁶), micro RNA will be extracted using the established protocols, e.g. a protocol that modifies the commonly used RNeasy Micro kit (Qiagen). The quantity and quality of RNA is examined using Nanodrop and Agilent Bioanalyzer capillary electrophoresis, respectively. Aliquots of the prepared RNA are then dispensed into multiple wells of a qPCR microtiter plate, each containing a specific DNA probe immobilized to its surface. RNA:DNA hybrid detecting immuno-phage particles are then added to each plate, and detected by quantitative PCR as described. The detectable sensitivity, i.e. the highest dilution of the assayed cells at which the targeted micro RNA can still be detected using the immuno-phage technology, and compare this sensitivity to that of conventional quantitative real-time RT-PCR. For each microRNA species, its relative ratio to that of a housekeeping gene (U6 RNA) will be recorded.

If the amount of CLL cells in total blood white counts is low, we can use flow cytometry sorting to purify small numbers of neoplastic cells based on their unique immunophenotype (CD5+/CD19+) before testing. The high level of sensitivity due the signal amplification caused by our parallel PCR approach will allow the detection and quantification of micro RNA in very small amounts of tissue, such as those available from, for example, needle core biopsies of pancreatic or liver lesions.

In a further example, the feasibility of using the technology in a clinical setting can be examined using cryo-preserved cells. They are thawed using an established protocol, and then the neoplastic B-cells (CD5+/CD19+) and the normal B-cells (CD5−/CD19+, as controls to normalize the expression of miRNA in neoplastic B-lymphocytes for each individual) from the same individual are selected using flow cytometry sorting by FACSAria. The use of normal B-cells from each individual controls for changes of micro RNA expression related to age, gender, marrow micro-environment, storage status and processing. Additionally, the amount of normal B-cells in B-CLL samples can be very small and will need a highly sensitive method as described to measure the micro RNA expression. The micro RNA is extracted from sorted neoplastic and normal B-cells using the protocol described above. Individual wells of a microtiter plate are spotted with DNA oligonucleotides complimentary to each of the target miRNAs The targeted miRNA in the neoplastic and normal B-cells is detected using the immuno-phage parallel amplification technology, and also the current standard of quantitative real-time PCR based assays using the same amounts of input RNA in the two cell populations. The ratio of expression of each miRNA between normal and neoplastic B-cells in each patient using both assays can be calculated to determine which miRNAs are over- or under-expressed in neoplastic lymphocytes. To further compare the detectable sensitivity (i.e. the minimal amount of cells which can be used to detect targeted micro RNAs) of the technology and the conventional quantitative real-time RT-PCR in clinical samples, we will perform serial 10-fold dilutions of the sorted cells as input down to as low as 500 cells.

In an effort to examine the clinical significance of the expression profiles of the targeted miRNA (which micro RNAs are up- or down-regulated) is correlated with the patient outcome.

### Example 4

### Detection, with High Specificity and Sensitivity, of Prognostic and Diagnostic NSC Lung Cancer Markers Using Novel Magnetic Immuno-Particles

In one example, the technology introduced here can be used for the ultrasensitive detection of protein and miRNA markers spiked into Fetal Bovine Serum. Protein targets are captured by specific polyclonal antibodies immobilized on a universal, solid surface with low non-specific binding and then recognized by the immuno-magnetic urchin particles decorated with a second, monoclonal antibody and amplifiable DNA labels. After extensive washing for enhanced specificity, the captured nanoparticles are eluted and detected using quantitative PCR. Micro RNAs can be detected as hybrids with specific, immobilized capture DNA probes, using urchin particles carrying an antibody specific for DNA/RNA hybrids.

Specifically, commercially-available paramagnetic particles, modified to contain controlled ratios of antibody and DNA labels can be used to capture specific analytes in complex media. Using 250 nm particles functionalized with a ratio of 500 DNA labels to 1 antibody, we currently can quantitate 4 to 40,000 particles by real-time PCR (FIG. 2).

In another example, we show the successful detection of a 15-mer miRNA mimic using immuno-magnetic urchins. A single-stranded DNA probe is immobilized in individual wells of a DNA-BIND™ PCR plate, coated with reactive N-oxysuccinimide esters. The complementary RNA (10³-10⁹ molecules per well) is then hybridized to the DNA capture probes at 50° C. for 12 hours, generating an RNA/DNA antigen recognized by the 250 nm immuno-magnetic urchins carrying the S9.6 anti RNA/DNA hybrid antibody and DNA labels. After five washes with PBS/Tween, the remaining urchins serve as templates for qPCR. As few as 1000 miRNA mimic molecules could be detected and the qPCR output was clearly proportional to the number of RNAs in the sample (FIG. 3, FIG. 11).

In another example, the following synthetic human miRNAs are detected: miR17-3p (5′-CAAAGUGCUUACAGUGCAGGUAGU-3′); miR21 (5′-UAGCUUAUCAGACUGAUGUUGA-3′), and miR106a (5′-AAAAGUGCUUACAGUGCAGGUAGC-3′) will be captured by complementary immobilized DNA probes.

In another example, the recombinant proteins SMAD and EGFR can be detected. EGFR is enzymatically phosphorylated in vitro using standard procedures. Confirmation of phosphorylation is done by western blotting and detection by ECL (GE Life Sciences, Piscataway, N.J.). For detection of proteins and phosphorylated proteins, we use a two-antibody sandwich assay format. Protein targets are captured by specific polyclonal antibodies immobilized on a universal, solid surface with low non-specific binding and then recognized by the immuno-magnetic urchin particles decorated with a second, monoclonal antibody. Smad1 (A-4) and Smad1 (H-2) antibodies are used for the detection of SMAD1. Anti-EGFR antibodies (C-20/N-20), anti-phospho-EGFR (pY1068) and/or anti-phospho-EGFR (pY1148) antibodies are used to detect phosphorylated and non-phosphorylated isoforms of EGFR.

Although the high-affinity biotin-streptavidin system has routinely been used for biomolecule immobilization, non-specificity issues have compromised assay sensitivity, and have not been fully resolved. In our current approach, we use discrete-length poly(ethylene) glycol (PEG) monolayers to inhibit non-specific biomolecule adsorption onto the surface and to act as a linker to capture ligands. More specifically, a mixed monolayer (FIG. 4) is formed using a mixture of long heterobifunctional PEG molecules with an active site for ligand attachment (e.g. NHS or maleimide for crosslinking between primary amines or sulfhydryl groups in proteins or nucleic acids) and an excess of short capped PEG molecules to reduce crowding and thus eliminate any steric hindrance effects in the layer of the immobilized ligand. The tethered molecules are highly active, behaving essentially as free molecules in solution (FIG. 12).

For any DNA amplification reaction, we will adopt the use of deoxyuracil (dUTP) and uracil-DNA glycosylase (UDG) in our amplification method. The incubation of PCRs with UDG prior to thermal cycling in combination with the use of dUTP in the PCR amplification is a commonly-used technology with established protocols to prevent contamination.

In other application of the technology introduced here, locked nucleic acid (LNA), a nucleic acid analogue containing one or more LNA nucleotide monomers with a bicyclic furanose unit locked in an RNA mimicking sugar conformation, can be an alternative to DNA probes. LNA oligonucleotides display unprecedented hybridization affinity toward complementary single-stranded RNA and complementary single- or double-stranded DNA and have been recently incorporated into biosensor applications.

In other application of the technology introduced here, aptamers, single stranded nucleic acids that are in vitro selected to recognize a variety of targets ranging from small molecules to proteins with extreme specificity might also be considered for the detection of protein analytes, as an alternative or complement to antibodies.

### Example 5

### Using Magnetic Force Discrimination to Markedly Increase Specificity and Decrease Background Noise

Even a highly sensitive assay is not useful if confounded by non-specific background. In this example, the magnetic properties of the particles are used to discriminate against any non-specifically bound particles prior to the quantitative amplification of their multiple DNA labels. A magnetic field is applied to test the strength of the bonds between the magnetic particles and the surface in order to “pull-off” any non-specifically bound particles that might lead to a false positive reading. Specifically, using hen egg lysozyme (HEL) and an anti-HEL IgG antibody we show that a Hall probe-calibrated horizontal magnetic force greater than 1000 pN is able to remove all bound particles, while a force of 200 to 250 pN gives the optimum discrimination between specifically and non-specifically bound particles. Neodymium permanent magnets are used in these preliminary experiments. The strength of the magnetic force exerted is tuned by changing the actual distance between the functionalized surface and the magnet.

In another modification of this application, to ensure reproducible magnetic forces, a sample mounting set up is engineered that allows tight control of that distance.

In another modification, an electromagnet whose strength is reproducibly and more easily tuned by changing the electric current is used.

### Example 6

### Detection of Protein Lung Cancer Biomarkers in Clinical Samples with Urchin Magnetic Immuno-Particles

In one application of the new technology, phosphorylated signaling proteins are detected. Many of the key survival and proliferation pathways activated in lung cancer cells are regulated through phosphorylation of key proteins, such as members of the epithelial growth factor receptor (EGFR) family and downstream effectors of this pathway. Using phospho-proteomic approaches, it has been shown specific changes in the phosphorylated isoforms of EGFR, HER2, IRS-1, SMAD and AKT in NSCLC tissue samples. In a study using immortalized human bronchial epithelial cells (HBECs), others demonstrated a general increase in protein phosphorylation in cells with mutant EGFR, including receptor tyrosine kinases (TKs) such as EGFR, HER2, MET, IGF1R and other regulators of the EGFR signaling pathway. Several other studies have demonstrated changes in patterns of protein phosphorylation of the EGFR-AKT-MAPK pathway in lung cancer and have shown possible diagnostic applications.

In this example, the urchin technology is used for detecting biomarkers in clinical samples which have a complex background of molecules. Specifically, we can measure ratios of phosphorylated vs. non-phosphorylated EGFR and SMAD proteins in human tissue samples from normal and NSCLC samples. Each sample is assayed with the urchin platform and with the standard qRT-PCR and ELISA reference assays. As a validation for our ability to prepare and detect protein against a complex sample background, defined amounts of external standard proteins (i.e. VEGF) are spiked into the tissue preparations at different concentrations. This allows the determination of the detection sensitivity, and the differences between benign and malignant lung tissue (ratio of phosphorylated vs. non-phosphorylated proteins, etc.). In this example, aliquots of the tissue are washed twice in ice-cold PBS, homogenized in stabilizing lysis buffer, centrifuged, and the supernatant is collected. Protein concentration is determined by routine assays. Real-time PCR is performed and the relative ratio of the target miRNAs to a housekeeping gene (U6 RNA) is recorded.

### Example 7

### Detection of miRNA Lung Cancer Biomarkers in Clinical Samples with Urchin Magnetic Immuno-Particles

Micro-RNAs (miRNAs), a class of small non-coding RNAs, are emerging as widely-important agents of post-transcriptional regulation of coding genes through mRNA decay and/or translational repression. miRNA networks have been proposed to represent one of the ‘hidden layers’ of regulation that integrates the transcriptome (the complete set of RNAs) of a cell with its proteome (the complete set of proteins). miRNAs bind to the 3′UTR of a target mRNA through a 7 bp seed region on the 5′ end, sequester the target mRNA in the RNA-induced Silencing Complex and mediate post-transcriptional gene silencing through deadenylation-mediated mRNA decay and/or translational suppression by mechanisms not yet fully elucidated. Micro RNAs have been associated with many aspects of development, differentiation, proliferation, apoptosis and virtually every important cellular process. There are currently more than 700 described human miRNAs in the miRBase with the potential for at least 1000 miRNAs based on sequence prediction. The dysregulation of miRNA expression has been documented in the vast majority of cancers, and contributes to tumor initiation and progression, and there is a significant interest in either targeting miRNAs or using them as prognostic and diagnostic targets, as well as therapeutic agents. It has recently been shown that levels of specific miRNAs can be used to distinguish normal from neoplastic respiratory cells. A study demonstrating diagnostic miRNA signatures of NSCLC revealed overexpression of 12 specific miRNAs (miR17-3p, miR21, miR106a, miR146, miR155, miR191, miR192, miR203, miR205, miR210, miR212, and miR214) when compared with normal lung tissue. Other studies confirmed miR21 overexpression in lung cancer, let-7 family and miR-20a down-regulation, as well as miR205 as a specific marker of squamous histology.

Specifically, we can measure levels of three lung cancer-associated miRNAs (miR-17-3p, miR-21 and miR-106a) in human tissue samples from normal and NSCLC samples. Each sample is assayed with the urchin platform and with the standard qRT-PCR and ELISA reference assays. As a validation for our ability to prepare and detect miRNA against a complex sample background, defined amounts of external miRNA are spiked into the tissue preparations at different concentrations. This allows the determination of the detection sensitivity, and the differences between benign and malignant lung tissue (miRNA abundance, ratios of different miRNAs). In this example, aliquots of the tissue are washed twice in ice-cold PBS, homogenized in stabilizing lysis buffer, centrifuged, and the supernatant is collected. Protein concentration is determined by routine assays. Real-time PCR is performed and the relative ratio of the target miRNAs to a housekeeping gene (U6 RNA) is recorded.

### Example 8

### Detection of Multiple miRNAs in the Same Sample

In an extension of the previous example, we show the ability to detect more than one biomarker in a parallel assay (including detection of miRNAs and proteins in the same sample). For multiple miRNA detection, separate wells of a microtiter plate are spotted with DNA oligonucleotides complementary to each of the three miRNAs we are examining initially (miR-17-3p, miR-21 and miR-106a). The DNA complement of the housekeeping U6 RNA which we will use as an internal standard to account for differences between individual sample preparations is spotted into a control well (Urchins alone are used as template to assure PCR functionality). Each sample of the prepared tissue material is screened for the presence of all the three miRNAs by adding sample aliquots into the corresponding, separate DNA wells. We can then detect DNA:miRNA hybrids using our urchin technology in combination with a magnetic discrimination step. The ratios of expression between miRNA and housekeeping RNA, both in benign and malignant samples, are calculated to determine which miRNAs are over- or under-expressed. In a parallel experiment, the sensitivity (i.e. the minimum amount of cells which can be used to detect targeted micro RNAs) of the urchin technology for clinical samples is determined by performing serial dilutions of the original samples into fetal bovine serum and assaying for an output (qPCR signal) from the urchin assay. All samples are tested at least ten times. Binary logistic regression and linear discriminant analysis (LDA) are used to identify significant predictors of benign and neoplastic lung tissue.

### Example 9

### Detection of Multiple Proteins in the Same Sample

For the parallel detection of multiple protein markers in the same sample, we employ urchin particles with unique DNA reporter sequences. A single surface is coated with specific antibodies for the protein targets of interest (e.g. EGFR, SMAD, EGFR-P, SMAD-P). After exposure to the sample, a mix of four discrete urchin particles bearing different DNA labels is used to bind to the bound protein targets. Magnetic force discrimination is used to enhance specificity and reduce background. In the qPCR four unique primer sets are used to probe for the abundance of each urchin.

### Example 10

### Ultra-Sensitive Multiplex Detection of Lung Cancer Biomarkers Using Immuno-Magnetic Particles

The proposed work aims to develop and evaluate a detection system based on novel immuno-magnetic nanoparticles, for the highly-specific multiplex detection of non-small cell lung cancer (NSCLC) biomarkers. In order to allow the sensitive and simultaneous detection of these biomarkers, we have developed “urchins”, which are immuno-magnetic nanoparticles decorated with target-specific antibodies and unique, amplifiable, multiplexable DNA reporting labels. We propose to detect multiple biomarkers (both miRNA and protein) with high sensitivity in the same sample using urchins along with magnetic force discrimination to significantly reduce non-specific background binding. We will test this method for the detection of NSCLC biomarkers. Early detection of NSCLC is correlated with a significantly better outcome for patients, and several studies have reported the ability to distinguish lung cancer from normal tissue based on expression of specific miRNAs or protein molecules. The sensitivity and specificity of current detection systems and single marker assays, however, precludes their clinical usefulness.

Particles are decorated with antibodies and a DNA reporter sequence, complementary to a 5′-nuclease probe. Each probe contains a unique fluorophore, proximal to a specific quencher molecule. Prior to polymerization Taq polymerase hydrolyzes the probe and the now fluorescent dye is released. Multiple different antibodies are immobilized on the surface of a qPCR plate, each recognizing a different target protein. Binding of the target subsequently retains the urchins, each carrying a unique fluorophore. Micro RNA hybridizes to a complementary DNA sequence immobilized on the surface, and is recognized by urchins containing a unique RNA:DNA specific antibody. During real-time PCR the fluorescent signal from multiple different urchins is recorded simultaneously (FIG. 13).

Multiplex detection including that of phosphorylated and non-phosphorylated protein variants, will be achieved in the same sample using urchins decorated with different antibodies and unique fluorophores. For the parallel detection of multiple targets in the same sample, it is important to emphasize that different urchin can carry different probes with different fluorophores. The DNA labels on each urchin type will be amplified with a different set of primers and a uniquely-labeled probe that will distinguish each PCR amplicon. For instance, an urchin specific for EGFR would be labeled with 6-FAM (emission wavelength=520 nm), while a CYFRA-specific urchin could be labeled with Cy5 (emission wavelength=668 nm). Numerous fluorophores are commercially available from several vendors.

In multiplex detection each well will be coated with antibodies against the target proteins, plus a single-stranded DNA molecule to capture a specific miRNA target. Equal volumes of samples and a mixture of target specific urchins are added to each well, and the reaction is monitored in a real-time PCR instrument. Targets are identified by the unique fluorophore on their specific urchin, and the position on the microtiter plate (FIG. 14).

### Example 11

### Ultra-Sensitive Detection of Proteins Using Silver Deposition and SEM

An antibody recognizing a protein biomarker is immobilized on conductive indium tin oxide in a microfluidic device by TESBA silane chemistry. A sample potentially containing the biomarker protein is contacted with the surface, which is then washed and contacted with a biotinylated second polyclonal antibody to the protein. After washing, the surface is contacted with neutravidin conjugated to 1.4 nm gold nanoparticles, washed, and treated with 8 mM Ag-acetate and 40 mM hydroquinone to deposit silver around the nanoparticles. The surface is then washed with water, ethanol and acetone, dried, and examined under 45 degree tilted SEM to enhance contrast and distinguish between surface-grown deposits and settled contaminant particles using a rapid lock-hopper device to increase processing speed. Silver spots are counted using ImageJ software, and after comparison to and normalization by controls and housekeeping reference proteins, the concentration of the biomarker is inferred and used to diagnose disease.

### Example 12

### Ultra-Sensitive Detection of Proteins Using Electrophoretic Pushoff, Silver Deposition and Environmental SEM

An antibody recognizing a protein biomarker is immobilized on an oxidized conductive silicon surface in a microfluidic device by APTES/glutaraldehyde silane chemistry. A sample potentially containing the biomarker protein is contacted with the surface, which is then washed and contacted with a biotinylated second polyclonal antibody to the protein. After washing, the surface is contacted with neutravidin conjugated to 1.4 nm gold nanoparticles and polyglutamic acid, washed, and then an electrical potential is applied between the ITO and a counterelectrode mounted beside it to remove some non-specifically bound proteins and nanoparticles by lateral electrophoretic force. The surface is then treated with 4 mM Ag-acetate and 40 mM hydroquinone to deposit silver around the nanoparticles. The surface is then washed with water and examined under an environmental SEM using an automated sample changer to increase processing speed. Silver spots are counted using ImageJ software, and after comparison to and normalization by controls and housekeeping reference proteins, the concentration of the biomarker is inferred and used to diagnose disease.

### Example 13

### Ultra-Sensitive Detection of Viruses Using Centrifugal Rolloff and Cathodoluminescence

An antibody recognizing a pathogenic virus is immobilized on conductive indium tin oxide in a microfluidic device by APTES silane chemistry. A sample potentially containing the virus is contacted with the surface, which is then washed and contacted with a biotinylated second polyclonal antibody to the virus. After washing, the surface is contacted with neutravidin conjugated to 200 nm Mn-doped ZnS phosphor nanoparticles, washed and left wet, and then placed in a centrifuge holder and exposed to 400,000 xg parallel to the surface to remove non-specifically bound particles. After the centrifugal treatment, the sample is washed, dried, and placed in a scanning electron microscope with a prism spectrometer and sensitive photomultiplier tube. Scanning of the surface with the electron beam produces flashes of cathodoluminescence which are counted and used to identify the presence of the virus in the sample.

### Example 14

### Detection of Motile Pathogens by Particle Tracking

A stool sample from a human suspected of being infected with Giardia is filtered and treated with a fluorescently-labeled antibody to that pathogen and imaged over time with video fluorescence microscopy. The video time series images are analyzed with computational tracking algorithms for pathogens involving two key routines applied in sequence: a pathogen-identifying routine, and a pathogen-tracking routine. In the first step, all pathogens in an individual image are automatically identified using spatial filtering and brightness thresholds, and each pathogen's shape and size are calculated. Spurious features whose shape and size are not characteristic of pathogens are eliminated. In the second step, suspected pathogen positions in consecutive frames are linked into trajectories by minimizing the total displacement of all pathogens. Individual trajectories are analyzed for statistical properties including speed, persistence length, and turning radius, and the presence of at least 100 trajectories per sample with characteristics typical of Giardia motion is interpreted as evidence of Giardia infection.

### Example 15

### Detection of Motile Pathogens by Particle Tracking after Hatching

A stool sample from a human suspected of being infected with Cryptosporidum is filtered and treated with 1% sodium taurocholate at 37 C for 20 h under 10% carbon dioxide to induce the excystation of Cryptosporidium oocysts, and then treated with a fluorescently-labeled antibody to that pathogen and subjected to video fluorescence microscopy, and the video images are subjected to computational tracking algorithms for pathogens involving two key routines applied in sequence: a pathogen-identifying routine, and a pathogen-tracking routine. In the first step, all pathogens in an individual image are automatically identified using spatial filtering and brightness thresholds, and each pathogen's shape and size are calculated. Spurious features whose shape and size are not characteristic of pathogens are eliminated. In the second step, suspected pathogen positions in consecutive frames are linked into trajectories by minimizing the total displacement of all pathogens. Trajectories are analyzed individually and trajectories exhibiting motion faster than diffusion, as determined by calculating the mean-square displacement versus time, are identified as evidence of Cryptosporidum infection.

### Example 16

### Detection of Motile Pathogens by Particle Tracking and Chemotaxis

A stool sample from a human suspected of being infected with Vibrio cholerae is filtered, treated with a fluorescently-labeled antibody to that pathogen, placed adjacent to a source of nutrients to create a gradient of nutrients toward which V. cholerae exhibits chemotaxis, and imaged over time with video fluorescence microscopy. The video time series images are analyzed with computational tracking algorithms for pathogens involving two key routines applied in sequence: a pathogen-identifying routine, and a pathogen-tracking routine. In the first step, all pathogens in an individual image are automatically identified using spatial filtering and brightness thresholds, and each pathogen's shape and size are calculated. Spurious features whose shape and size are not characteristic of pathogens are eliminated. Coordinated motion towards the source region of interest is identified by correlating the displacement vectors of neighboring pathogens as a function of time and spatial position.

Coordinated motion of immunofluorescently labeled features is automatically detected and interpreted as evidence of infection with V. cholerae.

### Example 17

### Detection of Motile Pathogens by Motion Vector Analysis and Chemotaxis

A stool sample from a human suspected of being infected with V. cholerae is filtered, treated with a fluorescently-labeled antibody to that pathogen, placed adjacent to a source of nutrients to create a gradient of nutrients toward which V. cholerae exhibits chemotaxis, and imaged over time with video fluorescence microscopy. Motion vector analysis is applied to each time series to identify the collective motion of pathogens towards a particular direction: the displacement for co-moving regions of an image (for example, rectangles) is calculated by maximizing the cross-correlation between corresponding regions in consecutive images of pathogens in a time series. Coordinated motion of immunofluorescently labeled features is automatically detected and interpreted as evidence of infection with V. cholerae.

### Example 18

### Detection of Toxins using Charge-Mutant Immunophage with Electrophoretic Binding and Pulloff

The wells of a microtiter plate are coated at the bottom with gold by vacuum deposition, and then with a 300 um layer of agarose. Antibodies to ricin toxin are immobilized on the agarose by periodate activation. An environmental sample suspected of being contaminated with ricin toxin is placed in a well of the microtiter plate, and the toxin is allowed to bind by diffusion, and the well washed. A phage mutant selected by repeated electrophoresis for its enhanced surface charge is used to construct immunophage bearing 55 copies of a PCR-amplifiable sequence in their genomes and also displaying camelid antibodies to ricin toxin. These phage are added to the well, and electrophoretically driven to the lower surface by applying a potential between the gold at the bottom and a counterelectrode near the top of the liquid layer. The bottom contact and the upper electrode are both provided by a robotically-inserted probe which provides both. The well is washed, fresh liquid is added, the probe is reinserted, and the voltage is applied with reversed polarity and a lower voltage to remove nonspecifically-bound phage particles. The liquid is removed, eluant liquid is added, and the liquid in the well is transferred to a qPCR tube for phage lysis and analysis. Early amplification of the marker sequences is interpreted as evidence of the presence of ricin toxin.

### Example 19

### Detection of Cryptosporidium Cells by Affinity Adsorption to a Rolling Surface

A stool sample from a human potentially positive for infection with Cryptosporidium is treated with a mixture of 250 nm paramagnetic particles, and fluors, each conjugated to anti-cryptosporidium antibodies. The sample is introduced to a rolling chamber in the presence of a magnetic field gradient sufficient to apply a downward force of 20 pN on each particle, as measured at the bottom surface of the chamber. Fluorescence microscopic observation focused at the bottom of the chamber reveals the presence of fluorescent objects which tend to remain at the bottom of the chamber and, at intervals, to move along the surface more slowly than the overall liquid flow rate. A smaller number of such objects is observed in a parallel channel lacking the magnetic field, and these results are interpreted as evidence for cryptosporidium infection.

### Example 20

### Detection of Cryptosporidium Cells by Affinity Adsorption to a Coulter Pore Surface

A stool sample from a human potentially positive for infection with Cryptosporidium is centrifuged, filtered, diluted, and passed through a 40 micron pore across which an electrostatic potential difference is applied and the resulting current is continuously measured. A duplicate sample is passed through an identical pore whose inner surface has been decorated with anti-cryptosporidium antibodies. The signal from the second pore contains events of reduced current and extended duration, which are interpreted as evidence for cryptosporidium infection.

### Example 21

### Detection of Cryptosporidium Cells by Affinity Adsorption to a Cantilever Channel Surface

A stool sample from a human potentially positive for infection with Cryptosporidium is centrifuged, filtered, diluted, and passed through a 30 by 30 micron channel running through a vibrating silicon cantilever. A duplicate sample is passed through an identical pore whose inner surface has been decorated with anti-cryptosporidium antibodies. The signal from the second channel contains events of an extended duration, which are interpreted as evidence for cryptosporidium infection.

### Example 22

### Detection of miRNA by Affinity Adsorption to a Cantilever Channel Surface

A total RNA preparation from a tumor biopsy specimen is mixed with 50 nm gold nanoparticles coated in poly(ethylene) glycol chains, some of which are terminated with DNA oligonucleotides complimentary to a miRNA of diagnostic interest. The particle suspension is passed through an array of 600, 5 micron by 5 micron channels running through vibrating silicon cantilevers, 400 of which have been decorated on their interior walls with an antibody specific to RNA/DNA hybrids using silane chemistry, and 200 of which bear BSA on their internal surfaces. The signals from the 400 channels with antibodies contains events of an extended duration and an implied mass increment corresponding to a single 50 nm gold particle; these events are much less common in the unconjugated channels and are used to infer the concentration of the diagnostic miRNA.

### Example 23

### Detection of miRNA by Affinity Adsorption to a Cantilever Channel Surface with Electrophoretic Rolloff

A total RNA preparation from a tumor biopsy specimen is mixed with 200 nm latex nanoparticles coated in poly(ethylene) glycol chains, some of which are terminated with DNA oligonucleotides complimentary to a miRNA of diagnostic interest. The particle suspension is passed through a 5 micron by 5 micron channel running through a vibrating silicon cantilever which has been decorated on its interior walls with an antibody specific to RNA/DNA hybrids using silane chemistry. The signal from the channel with antibodies contains events of a duration longer than expected from the fluid flow rate and an implied mass increment corresponding to a single 200 nm latex particle. After each such event has lasted for 2 seconds, an electrical potential is applied through the channel to electrophoretically remove the bound particle. These events are used to infer the concentration of the diagnostic miRNA.

### Example 24

### Detection of miRNA by Hybridization Adsorption to a Cantilever Channel Surface with Phage Reporter

A total RNA preparation from a tumor biopsy specimen is passed through a 5 micron by 5 micron channel running through a vibrating silicon cantilever which has been decorated on its interior walls with a DNA probe complimentary to a miRNA of diagnostic significance. The RNA preparation is followed by a suspension of phage particles bearing an antibody scFv fragment specific to RNA/DNA hybrids, and an increase in mass in the cantilever is used to determine the concentration of the miRNA.

### Example 25

### Detection of Anti-RVFV Antibodies by Serum Adsorption to a Cantilever Channel Surface with Phage Reporter Competition

A serum sample from a patient suspected of being infected with Rift Valley Fever Virus is passed through a 5 micron by 5 micron channel running through a vibrating silicon cantilever which has been decorated on its interior walls with RVFV coat proteins. The serum sample is followed by a landscape M13 phage bearing scFv antibodies to RVFV. Failure of the phage to bind is interpreted as evidence of RVFV-seropositivity.

### Example 26

### Detection of miRNA by Two-Stage Mass Analysis with Intermediate Density Change

A total RNA preparation from a tumor biopsy specimen is mixed with 200 nm porous silica nanoparticles coated in poly(ethylene) glycol chains, some of which are terminated with DNA oligonucleotides complimentary to a miRNA of diagnostic interest. The particle suspension is mixed with 3 nm gold nanoparticles conjugated to an antibody specific to RNA/DNA hybrids. The particle suspension is then passed in a flowing liquid stream through a 3 micron by 3 micron channel running through a vibrating silicon cantilever to determine the buoyant mass of the particles, and then though a tee inlet which adds an equal volume of buffered deuterium oxide, and finally through a second 3 micron by 3 micron channel running through a vibrating silicon cantilever to determine the buoyant mass of the particles in the denser liquid. The order of particles is preserved through the two cantilevers and the tee, and the densities measured for each particle in the two media are analyzed together to determine the gold loading of each particle, and the results are used to estimate the concentration of the miRNA in the original sample.

### Example 27

### Detection of Protein by Antibody Staining, and Coulter Detection

A protein preparation from a water sample is contacted with highly monodisperse 30 nm gold particles bearing antibodies to a toxin, and then with second antibodies to the toxin, and secondary antibodies to those antibodies, and analyzed in a Coulter principle device. Changes in the apparent size of the particles are interpreted as evidence of the presence of the toxin.

### Example 28

### Detection of Protein by In Situ PCR, Antibody np Staining, and Coulter Detection

A DNA preparation from a water sample is contacted with highly monodisperse 300 nm gold particles bearing DNA probes to a gene encoding a toxin. Bridge PCR with the immobilized probes is used to amplify a portion of the target gene, with the products remaining bound to the particles. The particles are mixed with 20 nm polystyrene latex particles bearing antibodies to double-stranded DNA, and the mixture analyzed by a Coulter principle device in which reduction of current is used to detect gold particles bearing polystyrene.

### Example 29

### Packed Cell Volume with Gold Nanoparticles

A sample of blood from a human positive for HIV infection is mixed with 250 nm gold particles bearing antibodies to CD4-positive white blood cells, in a vessel of the shape of a packed-cell-volume centrifuge tube with a narrow extension on the bottom. After settling, the layers in the narrow extension include contaminants, cell-free particles, and CD4-positive cells settled by the particles. The depth of the CD4-positive cell layer can be interpreted to infer the concentration of CD4 cells in the blood sample.

### Example 30

### Detection of CD4 Cells by Settling Packed Cell Volume with Gold Nanoparticles

A sample of blood from a human positive for HIV infection is mixed with 250 nm gold particles bearing antibodies to CD4-positive white blood cells, in a vessel of the shape of a packed-cell-volume centrifuge tube with a narrow extension on the bottom. After settling, the layers in the narrow extension include contaminants, cell-free particles, and CD4-positive cells settled by the particles. The depth of the CD4-positive cell layer can be interpreted to infer the concentration of CD4 cells in the blood sample.

### Example 31

### Detection of CD4 Cells by Flotation Packed Cell Volume with Microbubbles

A sample of blood from a human positive for HIV infection is mixed with 0.5 um buoyant microbubbles bearing antibodies to CD4-positive white blood cells, in a vessel of the shape of a packed-cell-volume centrifuge tube but inverted such that the narrow extension is on top. After settling, the layers in the narrow extension include air, cell-free microbubbles, and CD4-positive cells floated by microbubbles. The depth of the CD4-positive cell layer can be interpreted to infer the concentration of CD4 cells in the blood sample.

### Example 32

### Detection of CD4 Cells by Magnetic Packed Cell Volume

A sample of blood from a human positive for HIV infection is mixed with 1 um paramagnetic particles, and the cell are magnetically drawn into a 300 micron microfluidic passage with a 1 micron exit hole, to form a detectable packed bed of CD4 cells, where other cells can be discriminated against by their lesser magnetic mobility and visual appearance, and magnetic particles not bound to cells can be discriminated against by the visual appearance of the layer of particles, which differs from the appearance of the layer of cells.

### Example 33

### Detection of CD4 Cells by Settling Packed Cell Volume with Gold Nanoparticles and Silver Deposition

A sample of blood from a human positive for HIV infection is mixed with 2 nm gold particles conjugated to antibodies to CD4-positive white blood cells, in a vessel of the shape of a packed-cell-volume centrifuge tube with a narrow extension on the bottom. Silver is deposited around the nanoparticles by hydroquinone reduction of a silver salt. After settling, the layers in the narrow extension include contaminants, cell-free particles, and CD4-positive cells settled by the silver/gold particles. The depth of the CD4-positive cell layer can be interpreted to infer the concentration of CD4 cells in the blood sample.

### Example 34

### Detection of microRNA Using FIA Capture, Anti-Hybrid Antibody, Enzyme Staining Intensification, and Coulter Counting

Total RNA purified from a sputum sample is hybridized to 60 nm gold particles bearing DNA sequences complimentary to a miRNA sequence of diagnostic interest in a flow-injection analysis apparatus. The particles are diafiltered, then contacted with an anti-RNA/DNA hybrid antibody conjugated to alkaline phosphatase and BCIP/NBT to deposit insoluble precipitate on particles bearing the miRNA. The particles are then characterized by Coulter counting through a nanotube, and the number and properties of larger, less-conductive particles in the population are interpreted as evidence of the concentration of the miRNA in the sample.

### Example 35

### Detection of Phosphorylated Protein Variant Using Capture, Anti-Phosphorylation Antibody, Click Intensification, and Coulter Counting

Total protein purified from a biopsy sample is contacted with 150 nm polymer particles bearing antibodies to a protein whose phosphorylated form is of diagnostic interest. A second antibody which recognizes the phosphorylation and which is conjugated to a terminal alkyne moiety is added and allowed to bind for 15 minutes. The particles are washed and exchanged into deaerated DMSO, and PEG-azide and CuSO₄ are added along with a reductant to carry out a copper(I)-catalysed 1,2,3-triazole forming reaction between the azide and the terminal alkyne on the secondary antibody. The particles are then characterized by Coulter counting through a nanotube, and the number and properties of larger, less-conductive particles in the population are interpreted as evidence of the concentration of the phosphorylated protein in the sample.

### Example 36

### Detection of Phosphorylated Protein Variant Using Capture, Anti-Phosphorylation Antibody, Polymerization Intensification, and Coulter Counting

Total protein purified from a biopsy sample is contacted with 150 nm gold particles bearing antibodies to a protein whose phosphorylated form is of diagnostic interest. A second antibody which recognizes the phosphorylation and which is conjugated to multiple biotins is added and allowed to bind for 15 minutes, followed by the biofunctional photoinitiator streptavidin-eosin isothiocyanate (SA-EITC). The particles are washed and exchanged into deaerated monomer solution, and illuminated, and visible-light surface photopolymerization takes place on the surface of the particles. The particles are then characterized by Coulter counting through a nanopore, and the number and properties of larger, less-conductive particles in the population are interpreted as evidence of the concentration of the phosphorylated protein in the sample.

### Example 37

### Ultra-Sensitive Detection of Proteins Using Negative Silver Deposition and SEM

An antibody recognizing a protein biomarker is immobilized on 50 nm gold spots on conductive indium tin oxide in a microfluidic device by SAM chemistry. A sample potentially containing the biomarker protein is spiked with 100 nm polystyrene particles bearing a second antibody to the biomarker and then contacted with the surface, which is then washed. After washing, the surface is treated with 8 mM Ag-acetate and 40 mM hydroquinone to deposit silver around the gold. The surface is then washed with water, ethanol and acetone, dried, and examined under 45 degree tilted SEM to enhance contrast and distinguish between surface-grown deposits and settled contaminant particles using a rapid lock-hopper device to increase processing speed. Silver spots are counted using ImageJ software, with a lower number of silver spots suggesting the presence of the biomarker, and after comparison to and normalization by controls and housekeeping reference proteins, the concentration of the biomarker is inferred and used to diagnose disease.

### Example 38

### Detection of Phosphorylated Protein Variant Using Capture, Anti-Phosphorylation Antibody, Solver Intensification, and Accusizer Counting

Total protein purified from a biopsy sample is contacted with 150 nm polymer particles bearing antibodies to a protein whose phosphorylated form is of diagnostic interest. A second antibody which recognizes the phosphorylation and which is conjugated to a 2 nm gold particle is added and allowed to bind for 15 minutes. The particles are washed and silver salts are added along with a reductant deposit silver around the gold on the secondary antibody. The particles are then characterized by light-scattering counting, and changes in the number and properties of the particles in the population are interpreted as evidence of the concentration of the phosphorylated protein in the sample.

### Example 39

### Detection of Protein Using Lateral-Flow, Magnetization, and Magnetometry

A liquid containing total protein purified from a biopsy sample is contacted with 150 nm polymer particles containing a low-Curie temperature material and bearing antibodies to a protein of diagnostic interest and applied to the upstream end of a dry lateral-flow wicking matrix. A second antibody which recognizes the protein is positioned downstream on the lateral-flow wicking matrix. After development, the lateral-flow material is heated above the Curie point of the low-Curie point material in the particles in the presence of a magnetic field, cooled, and then scanned for the presence of magnetic material at the position of the second, capture antibody.

### Example 40

A urine sample is centrifuged and the pellet resuspended and observed microscopically, with digital image analysis. Non-Brownian motion of some particles is used, which is ascribed to the presence of a bacterial infection. An antibiotic is added, and the motion is observed to change, supporting the conclusion that the infecting organism is susceptible to that antibiotic.

### Example 41

A 50 nm thick gold film on glass, bearing a regular array of 100 nm holes, is treated with a silane and an antibody recognizing a virus. A cerebrospinal fluid sample potentially containing that virus is contacted with the film in a microfluidic device, followed by washing with PBS containing Tween-20, then distilled water, then an alkaline phosphatase-conjugated polyclonal antibody to the same virus. Flowing of a reagent containing ascorbic acid phosphate and silver ions, with real-time imaging of the light passing through the holes from an LED on the dry side of the glass, shows blocking of the light from 10% (300) of the holes, which is interpreted as evidence of the presence of the virus.

### Table 1: Elements of Embodiments

The methods described above are further described by amplification of individual elements of embodiments of the methods.

### Utility of the Invention

The utility of the invention lies in fields such as Clinical Diagnosis; Prognosis, Pathogen discovery; Biodefense; Research; Adulterant Detection; Counterfeit Detection; Food Safety; Taxonomic Classification; Microbial ecology; Environmental Monitoring; Agronomy; and Law Enforcement.

### Analytes of Interest

Analytes of interest from specimens of interest—and for which elements of specimen preparation, analyte modification, particulate label composition, specificity enhancement, analyte/label contacting, and detection, are selected and directed-include genomic DNA, methylated DNA, specific methylated DNA sequences, messenger RNA, fragmented DNA, fragmented RNA, fragmented mRNA, mitochondrial DNA, viral RNA, microRNA, in situ PCR product, polyA mRNA, RNA/DNA hybrid, pathogen DNA, pathogen RNA, metabolite, metabolic intermediate, hormone, pathogen, virus, bacterium, fungus, organelle, biomarker, lipid, carbohydrate, protein, glycoprotein, lipoprotein, phosphoprotein, specific phosphorylated or acetylated variant of a protein, or viral coat proteins, Cell surface receptor, protein, nucleic acid, mRNA, genomic DNA, PCR product, cDNA, peptide, hormone, drug, spore, virus, SSU RNAs, LSU-rRNAs, 5 S rRNA, spacer region DNA from rRNA gene clusters, 5.8 S rRNA, 4.5 S rRNA, 10 S RNA, RNAseP RNA, guide RNA, telomerase RNA, snRNAs—e.g. U1 RNA, scRNAs, Mitochondrial DNA, Virus DNA, virus RNA, PCR product, human DNA, human cDNA, artificial RNA, siRNA, enzyme substrate, enzyme, enzyme reaction product, Bacterium, virus, plant, animal, fungus, yeast, mold, Archaea; Eukyarotes; Spores; Fish; Human; Gram-Negative bacterium, Y. pestis, HIV1, Bacillus anthracis, Smallpox virus, Cryptosporidium parvum, Chromosomal DNA; rRNA; rDNA; cDNA; mt DNA, cpDNA, artificial RNA, plasmid DNA, oligonucleotides; PCR product; Viral RNA; Viral DNA; restriction fragment; YAC, BAC, cosmid, hormone, drug, pesticide, digoxin, insulin, HCG, atrazine, anthrax spore, teichoic acid, prion, chemical, toxin, chemical warfare agent, pollutant, Genomic DNA, methylated DNA, messenger RNA, fragmented DNA, fragmented RNA, fragmented mRNA, mitochondrial DNA, viral RNA, microRNA, in situ PCR product, polyA mRNA, RNA/DNA hybrid, protein, glycoprotein, lipoprotein, phosphoprotein, specific phosphorylated variant of protein, virus, or a chromosome.

### Analyte Source

Analytes to be detected or quantitated may be in or isolated from cells, body fluids, tissues, a biopsy specimen, blood, serum, plasma, stool, saliva, sputum, CSF, lavage fluid, nasal wash, urine, cell lysate, circulating tumor cells, FNAB cells, FACS fraction, immunomagnetic isolate, air filtrate, FFPE slices, fresh-frozen specimens, drinking water, natural water, sea water, soil water, soil leachate, fresh tissue, frozen tissue, neutral formalin-treated tissue, a formalin fixed paraffin embedded tissue block, an ethanol-fixed paraffin-embedded tissue block, a blood sample, air filtrate, tissue biopsy, fine needle aspirate, cancer cell, surgical site, soil sample, water sample, whole organism, spore, genetically-modified reporter cells, animal or human body fluids (blood, urine, saliva, sputum, sperm, biopsy sample, forensic samples, tumor cell, vascular plaques, transplant tissues, skin, urine; feces, cerebrospinal fluid); agricultural products (grains, seeds, plants, meat, livestock, vegetables, rumen contents, milk, etc.); soil, air particulates; PCR products; purified nucleic acids, amplified nucleic acids, natural waters, contaminated liquids; surface scrapings or swabbings; animal RNA, cell cultures, pharmaceutical production cultures, CHO cell cultures, bacterial cultures, virus-infected cultures, microbial colonies, FACS-sorted population, laser-capture microdissection fraction, magnetic separation subpopulation, or an FFPE extract. It may be advantageous to sample only a portion of the sample source.

### Sample Pre-Treatment

Centrifugation, extraction, adsorption, protease, nuclease, partitioning, washing, de-waxing, leaching, lysis, amplification, denature/renaturation, electrophoresis, precipitation, germination, culturing, PCR, disintegration of tissue, extraction from FFPE, LAMP, NASBA, emulsion PCR, phenol extraction, silica adsorption, IMAC, filtration, affinity capture, capture from a large volume of a dilute liquid source, air filtration, surgical biopsy, FNA, flow cytometry, laser capture microdissection.

### Analyte Modification

Labeling, conjugation, methylation, esterification, dephosphorylation, phosphorylation, acetylation, deacetylation, methylation, demethylation, denaturation, conjugation, haloacetic acid modification, hatching, growth, excystation, passaging, culture, de-blocking, proteolysis, nuclease digestion, cDNA preparation, amplification, DNA ball preparation, clonal amplification, multiplication, charge enhancement, hybridization, antibody binding, adsorption, aptamer binding, photo-linking.

### Label Elements

Elements which can be part of, all of, associated with, or attached to labels include a nanoparticle, gold particle, silver particle, silver, copper, zinc, or other metal coating or deposit, polymer, drag tag, magnetic particle, buoyant particle, microbubble, metal particle, charged moiety, dielectrophoresis tag, silicon dioxide, with and without impurities (e.g., quartz, glass, etc.), poly(methylmethacrylate), polyimide, silicon nitride, gold, silver, quantum dot, CdS, carbon dot, a phosphor such as silver-activated zinc sulfide or doped strontium aluminate, a fluor, a quencher, polymer, PMMA, polystyrene, pellicular, Janus particle, scattering particle, fluorescent particle, phosphorescent particle, an orifice, an orifice smaller than 5 microns, and orifice smaller than 1 um, and orifice smaller than 100 nm, a mirrored, flake, sphere, cube, retroreflector, insulator, conductor, bar-coded or labeled particle, porous particle, pellicular particle, solid particle, nanoshells, nanorods, IR absorbers, microwave absorbers, microspheres, liposomes, microspheres, capsules or liposomes associated with IR or microwave absorbers, light- or microwave-openable capsules, containers or liposomes, analytes such as viruses, cells, parasites and organisms, minicells, nucleic acids, nucleating agents, polymerization initiators, photografting reagents, proteins, molecular recognition elements, linkers, self-assembled monolayers, PEG, dendrimers, charge modifiers, PEG, stabilizing coatings, magnetic materials, magnetic materials of Curie temperature below 200 C, enzyme, microbial nanowires, DNA including aptamer sequences, amplifiable DNA, repeated sequences of DNA, phage, phage modified for conductivity, fusions or conjugates of detectable elements with molecular recognition elements, anti-antibody aptamer, aptamer directed to antibody-binding protein, absorbed or adsorbed detectable compound, explosive, combustible material, lithergolic, hypergolic, heme, luciferin, a phosphor, an azide or terminal alkyne or other click chemistry participant.

### Molecular Recognition Element

Molecular recognition elements which can be part of, associated with, or attached to labels can be selected from the group consisting of an antibody, antibody fragment, antibody analog, affybody, camelid or shark antibody analog, nucleic acid, carbohydrate, aptamer, ligand, chelators, peptide nucleic acid, locked nucleic acid, backbone-modified nucleic acid, lectin, padlock probe, substrate, receptor, viral protein, mixed, cDNA, metal chelate, boronate, peptide, enzyme substrate, enzyme reaction product, lipid bilayer, cell, tissue, microorganism, yeast, bacterium, parasite, protozoan, virus, hormone, drug, anti-RNA/DNA hybrid antibody, mutS, anti-DNA antibody, anti-methylation antibody, or an anti-phosphorylation antibody.

### Amplification or Signal Enhancement Methods

Amplification or signal enhancement methods, which may act upon the analyte, a label, or a component of the label, include hatching, growth, PCR, solid-phase PCR, LATE, EATL, or hot-restart amplification, solid-phase RCA, silver staining, metal deposition or plating, nickel, copper or zinc deposition, gold particle growth, polymerization, particle binding, grafting, photografting, click chemistry, a copper(I)-catalysed 1,2,3-triazole forming reaction between an azide and a terminal alkyne.

### Specificity Enhancement

The specificity of detection of analytes may be enhanced through removal of non-specifically bound labels by chemical or physical means. Chemical means of removal include denaturants, temperature, acids, bases, osmolytes and solvents. Physical means of removal include force, vibration, buoyancy, centrifugation, sedimentation field-flow, magnetic force, electrophoretic force, dielectrophoretic force, sonication, and lateral force. Susceptibility to means of removal may be enhanced by incorporation of moieties particularly responsive to means of removal, such as charged or dense moieties for electrophoretic or sedimentation-based removal.

When cells, spores or viruses are used, specificity may also be enhanced by genetic selection for reduced nonspecific binding, or increased responsiveness to stringency forces, e.g., mutants with enhanced or reduced charge or hydrophobicity, or nonspecific affinity for molecular recognition elements used in assays.

### Readout Methods

Readout methods, which may act upon the analyte, a label, or a component of the label, include optical measurements of turbidity, scattering, staining, fluorescence, FRET, fluorescence lifetime, cathodoluminescence, light obscuration, obscuration of members of an array of light detecting elements such as photovoltaics, fibers, or CCD pixels, absorbance, cavity-ringdown absorbance, conductivity, Coulter counting, Coulter counting using a nanotube or nanopore, adsorption to the surface of a nanopore, microchannel or cantilever, multi-angle scattering, particle tracking, organism tracking, single-particle tracking, rolling-particle tracking, particle, colony, plaque or spot counting, microscopy, packed particle volume, and phosphorescence, autoradiography, as well as SEM, AFM and SPM, nucleic acid detection by hybridization, amplification, Taqman, qPCR, RCA, HDA, LCR, qPCR, RT-PCR, NASBA, LAMP, RCA, WGA, in situ PCR, in situ WGA, polony formation, sequencing, single-molecule sequencing, nanopore analysis, nanopore sequencing, single-molecule imaging, DNA ball formation, immunoassay, immunoPCR, proximity ligation assay, enzyme activity assay. Electrical and chemical analysis methods may also be applied to labels or portions thereof, including conductivity, electric birefringence, complex impedance, electrophoresis, MEMS electrophoresis, mass spectrometry, GC, LC, LC-MS, DMA, IMS, ICR, mobility analysis, electrochemical detection, plasmon resonance, electrochemiluminescence ELISA, and chemiluminescence ELISA, and the detection of reactions such as polymerization, enzyme reactions, luciferase, peroxidase, phosphatase, or an oxidase such as glucose oxidase, including enzymes encoded in a virus, spore or cell used as a label.

### Location of Analysis

The location of steps of the analysis, which may be used singly or in combination, include microtiter plates, tubes, the surfaces of particles or beads, nanowell arrays, flow injection analysis apparatus, PCR machines, microfluidic chips, conductive surfaces, temperature-controlled environments, pressure chambers, ovens, irradiation chambers, electrophoretic, field-flow and chromatographic apparatus, microscope stages, luminometers, Coulter principle devices, cantilever and FET sensors, vacuum chambers, electron optical apparatus, single-molecule detection apparatus, single-molecule fluorescence detection apparatus, surfaces bearing electrodes or pillars, emulsions, and robotic apparatus. In many cases it may be advantageous to perform more than one type of analysis in series, either fractionating a sample using or based on the results of one method before performing an additional method, or by interpreting together the results of multiple methods.

### CONVENTIONS AND INTERPRETATION

Specific details described herein, including what is stated in the Field of the Invention are in every case a non-limiting description and exemplification of embodiments representing concrete ways in which the concepts of the invention may be practiced. This serves to teach one skilled in the art to employ the present invention in virtually any appropriately detailed system, structure or manner consistent with those concepts. Reference throughout this specification to “an exemplary embodiment” means that a particular feature, structure, or characteristic described in connection with the embodiment is included in at least one exemplary embodiment of the present invention. Thus, the appearances of the phrase “in an exemplary embodiment” in various places throughout this specification are not necessarily all referring to the same embodiment. Furthermore, the particular features, structures, or characteristics may be combined in any suitable manner in one or more embodiments. It will be seen that various changes and alternatives to the specific described embodiments and the details of those embodiments may be made within the scope of the invention. It will be appreciated that one or more of the elements depicted in the drawings can also be implemented in a more separated or integrated manner, or even removed or rendered as inoperable in certain cases, as is useful in accordance with a particular application. Because many varying and different embodiments may be made within the scope of the inventive concepts herein described and in the exemplary embodiments herein detailed, it is to be understood that the details herein are to be interpreted as illustrative and not as limiting the invention to that which is illustrated and described herein.

As used herein, the use of the word “a” or “an” when used in conjunction with the term “comprising” (or the synonymous “having”) in the claims and/or the specification may mean “one,” but it is also consistent with the meaning of “one or more,” “at least one,” and “one or more than one.” In addition, as used herein, the phrase “connected to” means joined to or placed into communication with, either directly or through intermediate components.

The above disclosed subject matter is to be considered illustrative, and not restrictive, and the appended claims are intended to cover all modifications, enhancements, and other embodiments that fall within the true scope of the present invention, which to the maximum extent allowed by law, is to be determined by the broadest permissible interpretation of the following claims and their equivalents, unrestricted or limited by the foregoing detailed descriptions of exemplary embodiments of the invention.

1. A method of assaying an analyte comprising the steps of:
a. contacting the analyte with a plurality of viruses or cells, said viruses or cells each containing multiple copies of a DNA or RNA sequence capable of detection by means comprising nucleic acid hybridization or amplification, said viruses or cells also being associated with a molecular recognition agent capable of binding with the analyte;
b. separating viruses or cells which have bound with the analyte from viruses or cells which have not bound with the analyte;
c. detecting the multiple copies of a DNA or RNA sequence capable of detection by means comprising nucleic acid hybridization or amplification, by means comprising nucleic acid hybridization or synthesis; and
d. using the presence of the DNA or RNA sequence to infer the presence or concentration of the analyte.

2. The method of claim 1 wherein the binding of the analyte to the virus or cell relies upon moieties selected from the group consisting of an antibody, antibody fragment, antibody analog, affybody, camelid or shark antibody analog, nucleic acid, carbohydrate, aptamer, ligand, chelators, peptide nucleic acid, locked nucleic acid, backbone-modified nucleic acid, lectin, padlock probe, substrate, receptor, viral protein, cDNA, metal chelate, boronate, peptide, enzyme substrate, anti-RNA/DNA hybrid antibody, mutS, anti-DNA antibody, anti-methylation antibody, anti-phosphorylation antibody, avidin, biodin, neutravidin, or streptavidin, associated with the virus or cell by genetic expression for surface display, chemical conjugation, avidin-biotin recognition, or an azide or terminal alkyne or other click chemistry participant.

3. The method of claim 1 wherein the detection of the RNA or DNA sequence relies upon means selected from the group consisting of microscopy, phosphorescence, autoradiography, SEM, AFM and SPM, nucleic acid detection by hybridization, amplification, Taqman, RCA, HDA, LCR, qPCR, RT-PCR, NASBA, LAMP, RCA, WGA, in situ PCR, in situ WGA, LATE, EATL, hot-restart amplification, solid-phase RCA polony formation, sequencing, single-molecule sequencing, nanopore analysis, nanopore sequencing, single-molecule imaging, DNA ball formation, immunoassay, immunoPCR, or proximity ligation assay.

4. The method of claim 1 wherein the separation of the viruses or cells which have bound with the analyte from viruses or cells which have not bound with the analyte relies upon means selected from the group consisting of immobilized analyte, immobilized analyte analog, immobilized molecular recognition agent capable of binding analyte, competitor capable of binding analyte, competitor capable of binding molecular recognition agent, washing, fluid flow, electrophoresis, electrophoretic blotting, buoyant force, magnetic force, centrifugal force, or dielectric force.

5. The method of claim 1 wherein the viruses or cells have been modified or selected for modified surface charge, resistance to eluants, resistance to low or high pH, low non-specific binding, low binding to antibody proteins, fluorescence, expression of enzymes, or carriage of reporter genes.

6. The method of claim 1 wherein said viruses or cells each contain one or more copies of a DNA or RNA sequence encoding an enzyme or fluorescent protein, and are detected by detecting virus or cell growth, or by detecting the expression of the encoded enzyme or fluorescent protein.

7. The method of claim 1 wherein the cells or viruses comprise bacteria, yeast, filamentous phage, phage, spores, Bacillus subtilis, Escherichia coli, Chinese Hamster Ovary cells, Saccharomyces cerevisiae, or M13, T7, P22, or Lambda phage.

8. The method of claim 1 wherein the molecular recognition agent is associated with the cell or virus by genetic expression, fusion to a protein of the cell or virus, capture by another molecular recognition agent expressed by the cell or virus, chemical conjugation, chemical conjugation to a partner expressed by the cell or virus, click chemistry, click chemistry coupling to a partner biosynthetically presented on the surface of the cell or virus, coupling to biotin biosynthetically presented on the surface of the cell or virus, or indirect coupling to a moiety on the surface of the cell or virus.

9. The method of claim 1 wherein the multiple copies of a DNA or RNA sequence are present in at least 4 copies.

10. The method of claim 1 wherein the multiple copies of a DNA or RNA sequence are present in at least 15 copies.

11. The method of claim 1 wherein the multiple copies of a DNA or RNA sequence are present in at least 50 copies.

12. The method of claim 1 wherein the multiple copies of a DNA or RNA sequence are present in at least 500 copies.

13. The method of claim 1 wherein the multiple copies of a DNA or RNA sequence are of length at least 20 nucleotides.

14. The method of claim 1 wherein the multiple copies of a DNA or RNA sequence are of length at least 100 nucleotides.

15. A method of assaying an analyte comprising the steps of:
a. contacting the analyte with a plurality of viruses or cells, said viruses or cells each containing one or more copies of a DNA or RNA sequence encoding an enzyme or fluorescent protein, said viruses or cells also being associated with a molecular recognition agent capable of binding with the analyte;
b. separating viruses or cells which have bound with the analyte from viruses or cells which have not bound with the analyte;
c. growing the virus or cell under permissive conditions; and
d. using the detected sequences, growth or gene expression to infer the presence or concentration of the analyte.

16. The method of claim 15 wherein the separation of the viruses or cells which have bound with the analyte from viruses or cells which have not bound with the analyte relies upon means selected from the group consisting of immobilized analyte, immobilized analyte analog, immobilized molecular recognition agent capable of binding analyte, competitor capable of binding analyte, competitor capable of binding molecular recognition agent, washing, fluid flow, electrophoresis, electrophoretic blotting, buoyant force, magnetic force, centrifugal force, or dielectric force.

17. The method of claim 15 wherein the molecular recognition agent is associated with the cell or virus by genetic expression, fusion to a protein of the cell or virus, capture by another molecular recognition agent expressed by the cell or virus, chemical conjugation, chemical conjugation to a partner expressed by the cell or virus, click chemistry, click chemistry coupling to a partner biosynthetically presented on the surface of the cell or virus, coupling to biotin biosynthetically presented on the surface of the cell or virus, or indirect coupling to a moiety on the surface of the cell or virus.

18. A composition comprising a population of cells or viruses whose genomes contains either repeated sequences of length greater than 20 nucleotides or sequences encoding a detectable enzyme or fluorescent protein, a molecular recognition agent linked to the outer surface of the cells or viruses by genetic expression or chemical coupling, where the molecular recognition agent is encoded by a non-mutagenized set of nucleic acid sequences, and an analyte bound to the molecular recognition agent.

19. The composition of claim 18 wherein the repeated sequences are of length greater than 100 nucleotides, and the analyte bound to the molecular recognition agent also is bound to a particle or surface.

20. The composition of claim 18 wherein the cells or viruses comprise a mixture of RNA or DNA sequences encoding molecular recognition elements, with not more than 100 different sequences encoding molecular recognition elements present at a frequency of more than 0.01% of the population.


SEQUENCE LISTING (19 sequences)

SEQ ID NO: 1 - DNA, 17 bp, Artificial Sequence
  Description of Artificial Sequence Reverse primer used to generate anti-vascular endothelial growth factor aptamer through polymerase chain reaction
ccctgcactcttgtctg

SEQ ID NO: 2 - DNA, 18 bp, Artificial Sequence
  Description of Artificial Sequence Forward primer used to generate anti-vascular endothelial growth factor aptamer through polymerase chain reaction. This sequence is biotinylated at its 5? end.
caggatagagcttgactt

SEQ ID NO: 3 - DNA, 67 bp, Artificial Sequence
  Description of Artificial Sequence Template used to generate anti-vascular endothelial growth factor aptamer through polymerase chain reaction.
caggatagagcttgactttttttttttttttttttttttttcccgtcttccagacaagagtgcaggg

SEQ ID NO: 4 - DNA, 158 bp, Artificial Sequence
  Description of Artificial Sequence Template 1 used to generate reporter sequence one through polymerase chain reaction. Is subsequently attached to immuno-magnetic particle.
ggaaggatccgctagcgtcgacgccacagaaagaagcgagctaaccctactatgcatccaagagagcccaagagagcagcaggcagcagcgaatctaacgtttagtttccctttcgacgatcgaccgatcgaaaggctcgagtctagaagatctggaa

SEQ ID NO: 5 - DNA, 155 bp, Artificial Sequence
  Description of Artificial Sequence Template 2 used to generate reporter sequence two through polymerase chain reaction. Is subsequently attached to immuno-magnetic particle.
ggaaggatccgctagcgtcgacgattagtgcaccgatcaggaccctactatgaatccaagagaagggaaggggaggagaggagaggagggaggagggaggaggacacaccacccctcaatcgctgacgagaggctcgagtctagaagatctggaa

SEQ ID NO: 6 - DNA, 24 bp, Artificial Sequence
  Description of Artificial Sequence Fluorescent probe 1 hybridizing to Template 1 (#4). Carries 6-carboxyfluorescein dye at its 5?end and TAMRA (rhodamine) dye quencher at its 3? end.
agcccaagagagcagcaggcagca

SEQ ID NO: 7 - DNA, 24 bp, Artificial Sequence
  Description of Artificial Sequence Primer 1 binding to Template 1 (#4), allowing amplification of sequence region hybridizing to fluorescent probe 1 (#6)
acgccacagaaagaagcgagctaa

SEQ ID NO: 8 - DNA, 24 bp, Artificial Sequence
  Description of Artificial Sequence Primer 2 binding to Template 1 (#4), allowing amplification of sequence region hybridizing to fluorescent probe 1 (#6)
atcggtcgatcgtcgaaagggaaa

SEQ ID NO: 9 - DNA, 28 bp, Artificial Sequence
  Description of Artificial Sequence Fluorescent probe 2 hybridizing to Template 2 (#5). Carries Hexachlorofluorescein dye at its 5?end and Iowa Black Quencher dye at its 3? end.
aggagggaggagggaggaggacacacca

SEQ ID NO: 10 - DNA, 26 bp, Artificial Sequence
  Description of Artificial Sequence Primer 1 binding to Template 2 (#5), allowing amplification of sequence region hybridizing to fluorescent probe 2 (#9)
tagactcgagcctctcgtcagcgatt

SEQ ID NO: 11 - DNA, 26 bp, Artificial Sequence
  Description of Artificial Sequence Primer 2 binding to Template 2 (#5), allowing amplification of sequence region hybridizing to fluorescent probe 2 (#9)
tgcaccgatcaggaccctactatgaa

SEQ ID NO: 12 - DNA, 18 bp, Artificial Sequence
  Description of Artificial Sequence Primer 12 used to generate reporter sequence one through polymerase chain reaction. Used with Template 1 (#4). Carries 5′ Amino Modifier with a C12 spacer.
ggaaggatccgctagcgt

SEQ ID NO: 13 - DNA, 18 bp, Artificial Sequence
  Description of Artificial Sequence Primer 13 used to generate reporter sequence one through polymerase chain reaction. Used with Template 1 (#4). Carries 5′ Biotin attachment.
ggaaggatccgctagcgt

SEQ ID NO: 14 - DNA, 18 bp, Artificial Sequence
  Description of Artificial Sequence Primer 14 used to generate reporter sequence one through polymerase chain reaction. Used with Template 1 (#4).
ggaaggatccgctagcgt

SEQ ID NO: 15 - DNA, 17 bp, Artificial Sequence
  Description of Artificial Sequence Primer 15 used to generate reporter sequence one through polymerase chain reaction. Used with Template 1 (#4).
ttccagatcttctagac

SEQ ID NO: 16 - DNA, 24 bp, Artificial Sequence
  Description of Artificial Sequence Primer 16 used to amplify Template 1 (#4) for diagnostic purposes.
atcggtcgatcgtcgaaagggaaa

SEQ ID NO: 17 - DNA, 24 bp, Artificial Sequence
  Description of Artificial Sequence Primer 17 used to amplify Template 1 (#4) for diagnostic purposes.
acgccacagaaagaagcgagctaa

SEQ ID NO: 18 - DNA, 26 bp, Artificial Sequence
  Description of Artificial Sequence Primer 18 used to amplify Template 2 (#5) for diagnostic purposes.
tagactcgagcctctcgtcagcgatt

SEQ ID NO: 19 - DNA, 26 bp, Artificial Sequence
  Description of Artificial Sequence Primer 19 used to amplify Template 2 (#5) for diagnostic purposes.
tgcaccgatcaggaccctactatgaa